(19) United States
(12) Patent Application Publication
(10) Pub. No.: US 2010/0080766 A1
(43) Pub. Date: Apr. 1, 2010

(54) EYELASH MAKEUP COMPOSITION AND PROCESS COMPRISING THE APPLICATION OF TWO COMPOSITIONS

(75) Inventors: Christophe DUMOUSSEAUX, Antony (FR); Carole LE MERRER, Montrouge (FR); Karl PAYS, Saint Maurice (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'OREAL, Paris (FR)

(21) Appl. No.: 12/564,122

(22) Filed: Sep. 22, 2009

Related U.S. Application Data
(60) Provisional application No. 61/104,306, filed on Oct. 10, 2008.

(30) Foreign Application Priority Data
Sep. 30, 2008 (FR) 08 56549

(51) Int. Cl. A61K 8/73; A61K 8/72; A61K 8/81; A61Q 1/10

(52) U.S. Cl. 424/70.13; 424/70.11; 424/70.16

(57) ABSTRACT

Composition containing a continuous aqueous phase, at least one silicone surfactant and at least one crosslinked polyelectrolyte. Process for making up or caring for keratin fibres, by applying to keratin fibres the noted composition and a second composition different from the first.

### REFERENCE TO PRIOR APPLICATIONS

This application claims priority to U.S. provisional application Ser. No. 61/104,306, filed Oct. 10, 2008; and to French patent application 08 56549, filed Sep. 30, 2008, both incorporated herein by reference.

### INTRODUCTION TO THE INVENTION

The present invention relates to a composition useful in particular for the non-therapeutic care or making up of keratin fibres (such as the eyelashes, the eyebrows or the hair), and also to a process comprising the application of this composition to keratin fibres.

The compositions used in the process according to the invention are preferably in the form of an eyelash product or mascara.

The term “mascara” means a composition intended to be applied to keratin fibres: it may be a keratin fibre makeup composition, a keratin fibre makeup base, or base coat, a composition to be applied over a mascara, also known as a top coat, or a cosmetic composition for treating keratin fibres. The mascara is more particularly intended for human keratin fibres, but also for false eyelashes.

To obtain a curling or lengthening effect on the eyelashes, it is known practice to use compositions that may be applied under the action of a source of heat. Very often, these compositions contain a film-forming polymer that is not water-soluble at room temperature or at the heating temperature. These compositions have the drawback of being difficult to remove with conventional products.

Similarly, aqueous compositions comprising a large amount (i.e. ≧5% solids) of film-forming polymer present in the form of particles in the aqueous phase (and thus water-insoluble) are often difficult to remove on account of the presence of the polymer.

One aim of the present invention is to provide a composition for facilitating the removal of compositions that may be applied with a source of heat, and also compositions comprising a large amount of film-forming polymer present in an aqueous phase in the form of particles.

Another aim of the present invention is to provide a process for coating keratin fibres that allows the production of a deposit on the eyelashes that shows good staying power over time, the deposit being readily removable.

Another aim of the present invention is to provide a process for coating keratin fibres that allows the production of a deposit on the eyelashes that has a good eyelash-lengthening effect, especially under the action of heat, and good staying power over time, the deposit being readily removable.

The inventors have discovered that the properties described above are obtained by using a first composition comprising a continuous aqueous phase, at least one crosslinked polyelectrolyte and at least one silicone surfactant. Optionally, it is also possible to use a second composition that may be applied under the action of a source of heat, or comprising a large amount of film-forming polymer present in an aqueous phase in the form of a dispersion.

### BRIEF DESCRIPTION OF THE FIGURES

FIG. 1 shows a kit comprising a mascara packaging and application assembly and a heating device separate from the packaging and application assembly.

FIG. 2 shows an applicator.

### DETAILED DESCRIPTION OF THE PREFERRED EMBODIMENTS

One subject of the invention is a composition comprising a continuous aqueous phase, at least one crosslinked polyelectrolyte and at least one silicone surfactant. Specifically, the use of a crosslinked polyelectrolyte and of a silicone surfactant makes it possible to improve the removal of this composition, but also of a second composition applied over the composition.

A subject of the invention is also a composition comprising a continuous aqueous phase, at least one nonionic silicone surfactant with an HLB of greater than or equal to 8 at 25° C., and at least one crosslinked polyelectrolyte chosen from acrylamide/sodium 2-acrylamidomethylpropanesulfonate copolymer, crosslinked sodium starch glycolate in powder form, crosslinked sodium polyacrylates, polyacrylic acid/alkyl acrylate copolymers, and crosslinked polyoxyethylenated AMPS/alkyl methacrylate copolymers, and mixtures thereof, and optionally also at least 5% by weight of film-forming polymer solids in the form of particles in dispersion relative to the total weight of the composition.

A subject of the invention is also a process for making up keratin fibres, comprising the application to the fibres of a composition as described above.

A subject of the invention is also a kit for the non-therapeutic care and/or making up of keratin fibres, especially the eyelashes or the eyebrows, comprising:
a first composition, especially for the non-therapeutic care or making up of keratin fibres, comprising a continuous aqueous phase, at least one crosslinked polyelectrolyte and at least one silicone surfactant, and
a second composition for making up and/or caring for keratin fibres.

A subject of the invention is also a kit for the non-therapeutic care and/or making up of keratin fibres, especially the eyelashes or the eyebrows, comprising:
a first composition comprising a continuous aqueous phase, at least one crosslinked polyelectrolyte and at least one silicone surfactant, and
a second composition for making up and/or caring for keratin fibres, the composition preferentially comprising a continuous aqueous phase, and at least one film-forming polymer in the form of particles in dispersion, the polymer being present in an amount at least equal to 5% of solids.

A subject of the invention is also, according to a particularly preferred embodiment, a kit for the non-therapeutic care and/or making up of keratin fibres, especially the eyelashes or the eyebrows, comprising:
a first composition comprising a continuous aqueous phase, at least one crosslinked polyelectrolyte and at least one silicone surfactant, and
a second composition for making up and/or caring for keratin fibres, the composition preferentially comprising at least one compound or a mixture of compounds, which, when the composition is brought to a temperature of greater than or equal to 40° C., gives the composition a spinnability dmax of greater than or equal to 5 mm, and
a device for applying the compositions; and/or heating means for bringing the second composition, prior to, simultaneously with or subsequent to its application, to a temperature of greater than or equal to 40° C.

The second composition may be brought to a temperature of greater than or equal to 40° C. prior to, simultaneously with or subsequent to its application to the first coat of first composition, especially using an application device comprising a heater, such as a heating brush, etc. Also useful herein is a “heating means” as specifically disclosed herein, and including its statutory equivalents. Notably, terms like “an application device comprising a heater” and the like are generic to any application device containing a heater, and are not a 112, 6^{th} paragraph terms.

A subject of the invention is also a process, preferably a non-therapeutic cosmetic process, for making up or caring for keratin fibres, comprising the application to the keratin fibres:
of a first composition comprising a continuous aqueous phase, at least one crosslinked polyelectrolyte and at least one silicone surfactant, and then
of a second composition, the second composition preferentially comprising at least one compound or a mixture of compounds, which, when the composition is brought to a temperature of greater than or equal to 40° C., gives the composition a spinnability dmax of greater than or equal to 5 mm,
   the second composition being brought, prior to, simultaneously with or subsequent to its application, to a temperature of greater than or equal to 40° C.

The spinnability represents the capacity of the composition, once subjected to a source of heat, to form on the keratin fibres threads which, after drawing with an applicator, are sufficiently consistent and keep their shape. The use of heat so as to bring the composition to a temperature of greater than or equal to 40° C. makes it possible to control the length of the threads formed in the prolongation of the eyelashes.

In particular, after application of the softened composition and drawing of the threads, they solidify at room temperature in the prolongation of each eyelash and enable a noteworthy lengthening effect to be obtained.

The application, prior to the second composition, of a first composition comprising a continuous aqueous phase, at least one crosslinked polyelectrolyte and at least one silicone surfactant makes it possible to form a first film on the eyelashes that withstands heat when the second composition is subjected to a source of heat. A first film (base film) is obtained on the eyelashes, which is less cohesive than the second film formed by the second composition on top of the base film, which facilitates the removal of the whole deposit on the eyelashes.

The second composition may be used especially in combination with a heating instrument, such as a heating brush, which may be applied to the eyelashes before, while or after they are coated with the composition or conditioned in a device allowing the composition to be applied while hot.

The second composition may be applied to all or to the top end of the keratin fibres, in particular the eyelashes. According to one embodiment, the second composition is applied to the top end of the eyelashes.

The first composition is preferably applied to the entire eyelash.

The first and second compositions comprise a physiologically acceptable medium, i.e. a non-toxic medium that may be applied to human keratin fibres, such as the eyelashes, the eyebrows and the hair, and that especially is compatible with the region of the eyes.

The first composition may be coloured (i.e. it may comprise at least one dyestuff as defined later) or uncoloured.

According to one embodiment, the second cosmetic composition is coloured, so as to form coloured threads at the end of the eyelashes.

According to another embodiment, the process according to the invention comprises:
applying a first uncoloured cosmetic composition to keratin fibres, the first composition comprising a continuous aqueous phase, at least one crosslinked polyelectrolyte and at least one silicone surfactant; and then
applying a second uncoloured cosmetic composition to the keratin fibres, the second composition preferably comprising at least one compound or a mixture of compounds, which, when the composition is brought to a temperature of greater than or equal to 40° C., gives the composition a spinnability dmax of greater than or equal to 5 mm,
bringing the second composition, prior to, simultaneously with or subsequent to its application, to a temperature of greater than or equal to 40° C., so as to form uncoloured threads at the end of the eyelashes,
and then, after cooling the threads, making up the keratin fibres and the uncoloured threads set in their prolongation with a third composition comprising one or more dyestuffs, so as to colour the threads.

In particular, the second composition comprising at least one compound or a mixture of compounds, which, when the composition is brought to a temperature of greater than or equal to 40° C., gives the composition a spinnability dmax of greater than or equal to 5 mm, is applied to the upper end of the keratin fibres, in particular the eyelashes.

I) First Composition

According to one embodiment, the first composition comprises a continuous aqueous phase, at least one crosslinked polyelectrolyte and at least one silicone surfactant.

a) Continuous Aqueous Phase

The first composition according to the invention comprises a continuous aqueous phase.

The term “composition with a continuous aqueous phase” means that the composition has a conductivity, measured at 25° C., of greater than 23 μS/cm (microSiemens/cm), the conductivity being measured, for example, using an MPC227 conductimeter from Mettler Toledo and an Inlab 730 conductivity measuring cell. The measuring cell is immersed in the composition so as to remove the air bubbles liable to form between the two electrodes of the cell. The conductivity reading is taken as soon as the conductimeter value has stabilized. A mean of at least three successive measurements is determined.

The aqueous phase may consist of, consist essentially of, or comprise water; it may also further consist essentially of, or further comprise, one or more water-miscible solvents (miscibility in water of greater than 50% by weight at 25° C.), for instance lower monoalcohols containing from 1 to 5 carbon atoms, such as ethanol or isopropanol, glycols containing from 2 to 8 carbon atoms, such as propylene glycol, ethylene glycol, 1,3-butylene glycol and dipropylene glycol, C₃-C₄ ketones and C₂-C₄ aldehydes, and mixtures thereof.

The aqueous phase (water and optionally the water-miscible solvent) may for example be present in a content ranging from 30% to 98% by weight, preferably ranging from 40% to 95% by weight and preferentially ranging from 40% to 90% by weight relative to the total weight of the composition comprising it.

b) Silicone Surfactant

The term “silicone surfactant” means a silicone compound comprising at least one oxyethylene chain. In a preferred embodiment the silicone surfactant comprises at least one oxyethylene (—OCH2CH2-) and/or oxypropylene (—OCH2CH2CH2-) chain.

According to the invention, a surfactant appropriately chosen to obtain a wax-in-water or oil-in-water emulsion is generally used. In particular, an emulsifier having at 25° C. an HLB (hydrophilic-lipophilic balance), in the Griffin sense, of greater than or equal to 8 may be used.

The HLB value according to Griffin is defined in J. Soc. Cosm. Chem. 1954 (volume 5), pages 249-256.

These silicone surfactants may be chosen from nonionic, anionic, cationic and amphoteric silicone surfactants or emulsifying surfactants. Reference may be made to the document “Encyclopaedia of Chemical Technology, Kirk-Othmer”, volume 22, pp. 333-432, 3rd edition, 1979, Wiley, for the definition of the properties and (emulsifying) functions of surfactants, in particular pp. 347-377 of this reference, for anionic, amphoteric and nonionic surfactants.

The silicone surfactants preferentially used in the compositions according to the invention are chosen from:

a) nonionic silicone surfactants with an HLB of greater than or equal to 8 at 25° C., used alone or as a mixture; mention may be made especially of:

polydimethylsiloxanes comprising both oxyethylene groups and oxypropylene groups. Examples that may be mentioned include the polydimethylsiloxane with oxyethylene/oxypropylene end groups sold as a mixture with caprylic/capric acid triglycerides under the name Abil Care 85 by the company Goldschmidt (INCI name: Bis-PEG/PPG-16/16 PEG/PPG-16/16 dimethicone/caprylic/capric triglyceride), the polydimethylsiloxane containing alpha-omega polyether groups (OE/OP:40/60), sold under the name Abil B8832 by the company Goldschmidt (INCI name: Bis-PEG/PPG-20/20 dimethicone), and the oxyethylenated oxypropylenated polydimethylsiloxane sold under the name Abil B88184 by the company Goldschmidt (INCI name: PEG/PPG-20/6 dimethicone),

the dimethicone copolyol of INCI name PEG/PPG-17/18 dimethicone, such as the product sold under the name Q2-5220 Resin Modifier® by the company Dow Corning,

dimethicone copolyol benzoate (Finsolv SLB 101° and 201® from the company Finetex),

and mixtures thereof;

b) amphoteric silicone surfactants such as dimethicone copolyol phosphates, such as the product sold under the name Pecosil PS100® by the company Phoenix Chemical;

c) and mixtures thereof.

According to one preferred mode, the silicone surfactant is chosen from polydimethylsiloxanes comprising both oxyethylene groups and oxypropylene groups, and dimethicone copolyol, and mixtures thereof.

More preferably, the silicone surfactant is chosen from polydimethylsiloxanes comprising both oxyethylene groups and oxypropylene groups.

According to one preferred embodiment of the invention, the silicone surfactant is Abil Care 85 or Q2-5220 Resin Modifier®.

The silicone surfactant may be present in a content ranging from 0.2% to 20% by weight, preferably from 0.5% to 15% by weight and better still from 1% to 10% by weight relative to the total weight of the first composition.

According to one particular mode, the silicone surfactant is present in a content of at least 0.2% by weight and preferably at least 5% by weight relative to the total weight of the composition. In particular, the silicone surfactant is present in a content ranging from 0.2% to 20% by weight and preferably from 0.5% to 15% by weight relative to the total weight of the composition.

c) Crosslinked Polyelectrolyte

The term “polyelectrolyte” means a macromolecular substance that has the capacity of dissociating when it is dissolved in water or in any other ionizing medium, to give at least one ion. In other words, a polyelectrolyte is a polymer comprising at least one ionizable monomer.

In particular, the polyelectrolyte may give polyions, for example polyanions, when it is dissociated in water. A polyelectrolyte may be a polyacid, a polybase, a polysalt or a polyampholyte. In the context of the invention, it is preferably a polyacid, and preferably a strong polyacid.

Preferably, the polyelectrolyte included in the first cosmetic composition according to the present invention is a crosslinked anionic polymer.

Preferably, the polyelectrolyte is also capable of forming a gel in solution at 0.5% by weight (of solids).

The counterions of the polyions formed during the dissociation may be mineral or organic, of any nature.

In particular, when the crosslinked polyelectrolyte is a crosslinked anionic polymer, the cations may be alkali metal or alkaline-earth metal cations such as sodium or potassium, or alternatively the ammonium ion.

The sodium cation Na⁺ is preferred, which is why it is mainly cited in the list of crosslinked polyelectrolytes that follows, without this constituting any limitation to this specific counterion.

Crosslinked polyelectrolytes that may be mentioned include:
the copolymer of acrylamide/2-methyl-2-[(1-oxo-2-propenyl)amino]-1-propanesulfonic acid (AMPS), partially or totally salified, especially in sodium salt form, such as Simulgel 600° in emulsion form containing polysorbate 80 as surfactant and containing isohexadecane as oil phase, sold by the company SEPPIC, or alternatively Simulgel EG®, Simulgel A® and Simulgel 501® sold by the same company.

Simulgel 600® is especially described in document FR 2 785 801. It is in effect an inverse latex. The AMPS polyelectrolyte is 2-methyl-2-[(1-oxo-2-propenyl)amino]-1-propanesulfonic acid partially or totally salified especially in sodium salt or ammonium salt form to a proportion of 30 mol % to 50 mol % in the mixture comprising AMPS and also an acrylamide, which is itself present in a proportion of 50% to 70%.
crosslinked sodium starch glycolate in powder form,
crosslinked sodium polyacrylates such as Norsocryl S35® sold by the company Atofina, or Cosmedia SP® sold by the company Cognis,
polyacrylic acid/alkyl acrylate copolymers of Pemulen® type,
AMPS (polyacrylamidomethylpropanesulfonic acid partially neutralized with ammonia and highly crosslinked) sold by the company Clariant,
crosslinked polyoxyethylenated AMPS/alkyl methacrylate copolymers,
mixtures thereof.

Crosslinked sodium polyacrylate and acrylamide/AMPS copolymer and copolymers thereof are most particularly suitable for use in the invention.

It will obviously be arranged for the crosslinked polyelectrolyte content to be adjusted such that the makeup-removing capacity is effectively improved, while at the same time not being detrimental to the water resistance of the cosmetic composition.

It is understood that the amount of crosslinked polyelectrolyte can vary significantly depending on the nature of the crosslinked polyelectrolyte. In general, this amount is at least equal to the amount that is necessary and sufficient to give the composition a better makeup-removing capacity. It is also termed the effective amount.

This makeup-removing capacity may especially be assessed by means of the test featured in Example 1.

Without this constituting any limitation to the invention, the inventors have put forward the theory that the crosslinked polyelectrolyte dispersed in the oil acts as a “water pump”. Thus, this “water pump” role is more clearly seen when the composition is placed in contact with an aqueous phase at the time of makeup removal. During makeup removal, the film of the cosmetic composition according to the invention is embrittled at the surface and results in its mechanical rupture; fragmentation of the film then takes place.

Preferably, the crosslinked polyelectrolyte is present in a content (of solids) ranging from 0.2% to 15% by weight, preferably from 0.5% to 10% and better still from 0.6% to 5% by weight relative to the total weight of the first composition.

According to one embodiment, the first composition comprises at least one film-forming polymer dispersed in particle form in the aqueous phase, the polymer being present in an amount at least equal to 5% of solids.

In this case, the first composition may be used without second composition. It is removed easily with a standard makeup remover.

d) Film-Forming Polymer Dispersed in Particle Form in the Aqueous Phase

In the present invention, the term “film-forming polymer” means a polymer that is capable, by itself or in the presence of an auxiliary film-forming agent, of forming a macroscopically continuous film that adheres to keratin fibres.

The film-forming polymer is present in the composition according to the invention in a solids (or active material) content at least equal to 5% by weight, preferably ranging from 5% to 40% by weight, better still from 7% to 30% by weight and better still from 10% to 25% by weight relative to the total weight of the first composition.

The film-forming polymer, when it is present, is dispersed in the composition in the form of particles in the aqueous phase, which is generally known as a latex or pseudolatex. The techniques for preparing these dispersions are well known to those skilled in the art.

Aqueous dispersions of film-forming polymer that may be used include the acrylic dispersions sold under the names Neocryl XK-90®, Neocryl A-1070®, Neocryl A-1090®, Neocryl BT-62®, Neocryl A-1079® 1090® and Neocryl A-523® by the company Avecia-Neoresins, Dow Latex 432® by the company Dow Chemical, Daitosol 5000 AD® or Daitosol 5000 SJ® by the company Daito Kasey Kogyo; Syntran 5760® by the company Interpolymer, Allianz Opt® by the company Rohm & Haas, aqueous dispersions of acrylic or styrene/acrylic polymers sold under the brand name Joncryl® by the company Johnson Polymer, or the aqueous polyurethane dispersions sold under the names Neorez R-981® and Neorez R-974® by the company Avecia-Neoresins, Avalure UR-405®, Avalure UR-410®, Avalure UR-425®, Avalure UR-450®, Sancure 875®, Sancure 861®, Sancure 878® and Sancure 2060® by the company Goodrich, Impranil 85® by the company Bayer, Aquamere H-1511® by the company Hydromer; the sulfopolyesters sold under the brand name Eastman AQ® by the company Eastman Chemical Products, vinyl dispersions, for instance Mexomer PAM® from the company Chimex, and mixtures thereof.

According to one advantageous embodiment, the composition according to the invention comprises at least one acrylic film-forming polymer in the form of solid particles dispersed in the aqueous phase, the polymer preferably resulting from the polymerization of at least one ethylenically unsaturated monomer chosen from α,β-ethylenic carboxylic acids, esters thereof and amides thereof.

α,β-Ethylenic unsaturated carboxylic acids that may be used include acrylic acid, methacrylic acid, crotonic acid, maleic acid and itaconic acid. (Meth)acrylic acid and crotonic acid are preferably used, and more preferentially (meth)acrylic acid.

The esters of these carboxylic acids may be chosen from the esters of (meth)acrylic acid (also known as (meth)acrylates), especially alkyl(meth)acrylates, in particular of a C₁-C₃₀ and preferably C₁-C₂₀ alkyl, aryl(meth)acrylates, in particular of a C₆-C₁₀ aryl, hydroxyalkyl(meth)acrylates, in particular of a C₂-C₆ hydroxyalkyl.

Among the alkyl(meth)acrylates that may be mentioned are methyl methacrylate, ethyl methacrylate, butyl methacrylate, isobutyl methacrylate, 2-ethylhexyl methacrylate, lauryl methacrylate and cyclohexyl methacrylate.

Among the hydroxyalkyl(meth)acrylates that may be mentioned are hydroxyethyl acrylate, 2-hydroxypropyl acrylate, hydroxyethyl methacrylate and 2-hydroxypropyl methacrylate.

Among the aryl(meth)acrylates that may be mentioned are benzyl acrylate and phenyl acrylate.

Needless to say, it is possible to use a mixture of these monomers.

The (meth)acrylic acid esters that are particularly preferred are the alkyl(meth)acrylates.

The alkyl group of the esters may be either fluorinated or perfluorinated, i.e., some or all of the hydrogen atoms of the alkyl group are replaced with fluorine atoms.

Amides of the carboxylic acids that may be mentioned, for example, include (meth)acrylamides, especially N-alkyl(meth)acrylamides and in particular of a C₂-C₁₂ alkyl. Among the N-alkyl(meth)acrylamides that may be mentioned are N-ethylacrylamide, N-butyl-acrylamide, N-octylacrylamide and N-undecylacrylamide.

The acrylic film-forming polymer that may be used according to the invention may comprise, in addition to the monomers mentioned above, at least one styrene monomer, such as styrene or α-methylstyrene.

Acrylic polymers that may be used include those sold under the names Syntran® 5190, Syntran® 5760 and Syntran® 5009 by the company Interpolymer, and Dow Latex 424® by the company Dow Chemical.

The first composition may also comprise additional compounds chosen from salts, water-soluble gelling agents, waxes, pasty compounds, non-silicone surfactants and dyestuffs.

e) Additional Compounds

Salts

The first composition according to the invention preferably comprises at least one salt.

Without this constituting any limitation on the invention, the inventors have put forward the hypothesis that the salt makes it possible to limit the swelling of the crosslinked polyelectrolyte in the composition.

As salts that may be used, mention may be made especially of monovalent, divalent or trivalent metal salts and more particularly alkaline-earth metal salts such as barium, calcium and strontium salts; alkali metal salts such as sodium and potassium salts, magnesium, beryllium, yttrium, lanthanum, cerium, praseodymium, neodymium, promethium, samarium, europium, gadolinium, terbium, dysprosium, holmium, erbium, thulium, ytterbium, lutetium, lithium, tin, zinc, manganese, cobalt, nickel, iron, copper, rubidium, aluminium, silicon and selenium salts, and mixtures thereof.

The ions constituting these salts may be chosen, for example, from carbonates, bicarbonates, sulfates, glycerophosphates, borates, chlorides, bromides, nitrates, acetates, hydroxides, persulfates and also the salts of α-hydroxy acids (citrates, tartrates, lactates or malates) or of fruit acids, or alternatively amino acid salts (aspartate, arginate, glycocholate or fumarate).

Preferably, calcium, magnesium, sodium or potassium salts and mixtures thereof are used, and more particularly magnesium chloride, potassium chloride, sodium chloride, calcium chloride and magnesium bromide, and mixtures thereof.

Preferably, the first composition comprises sodium chloride.

Preferably, the salt(s) is (are) present in a content ranging from 0.1% to 5% by weight, preferably from 0.5% to 10% and better still from 0.6% to 5% by weight relative to the total weight of the first composition.

Water-Soluble Thickeners

The first composition according to the invention may comprise a water-soluble thickener.

The water-soluble thickeners that may be used in the compositions according to the invention may be chosen from:

homopolymers or copolymers of acrylic or methacrylic acids or the salts and esters thereof, and in particular the products sold under the names Versicol F® or Versicol K® by the company Allied Colloid, and Ultrahold 8® by the company Ciba-Geigy;

copolymers of acrylic acid and of acrylamide sold in the form of the sodium salt thereof under the name Reten® by the company Hercules, sodium polymethacrylate sold under the name Darvan 7® by the company Vanderbilt, and the sodium salts of polyhydroxycarboxylic acids sold under the name Hydagen F® by the company Henkel;

proteins, for instance proteins of plant origin such as wheat proteins and soybean proteins; proteins of animal origin such as keratins, for example keratin hydrolysates and sulfonic keratins;

cellulose polymers such as hydroxyethylcellulose, hydroxypropylcellulose, methylcellulose and ethylhydroxyethylcellulose, and also quaternized cellulose derivatives;

acrylic polymers or copolymers, such as polyacrylates or polymethacrylates;

vinyl polymers, for instance polyvinylpyrrolidones, copolymers of methyl vinyl ether and of malic anhydride, the copolymer of vinyl acetate and of crotonic acid, copolymers of vinylpyrrolidone and of vinyl acetate; copolymers of vinylpyrrolidone and of caprolactam; polyvinyl alcohol;

polymers of natural origin, which are optionally modified, such as:

gum arabics, guar gum, xanthan derivatives, karaya gum;

alginates and carrageenans; glycosaminoglycans, hyaluronic acid and derivatives thereof;

shellac resin, sandarac gum, dammar resins, elemi gums and copal resins;

deoxyribonucleic acid;

mucopolysaccharides such as chondroitin sulfate,

and mixtures thereof.

According to one particular mode, the water-soluble thickener is chosen from cellulose polymers; hydroxyethylcellulose will preferably be used.

The water-soluble thickening polymer may be present in the composition comprising it in a solids content ranging from 0.01% to 60% by weight, preferably from 0.5% to 40% by weight, better still from 1% to 30% by weight, or even from 5% to 20% by weight, relative to the total weight of the composition comprising it.

Waxes

The wax under consideration in the context of the present invention is generally a lipophilic compound that is solid at room temperature (25° C.), which may or may not be deformable, with a solid/liquid reversible change of state, having a melting point of greater than or equal to 30° C., which may be up to 100° C. and in particular up to 90° C.

By bringing the wax to the liquid form (melting), it is possible to make it miscible with oils and to form a microscopically uniform mixture, but on cooling the mixture to room temperature, recrystallization of the wax in the oils of the mixture is obtained.

In particular, the waxes that are suitable for use in the invention may have a melting point of greater than or equal to 45° C., better still greater than or equal to 50° C. and even better still greater than or equal to 60° C.

For the purposes of the invention, the melting point corresponds to the temperature of the most endothermic peak observed by thermal analysis (DSC) as described in ISO standard 11357-3; 1999. The melting point of the wax may be measured using a differential scanning calorimeter (DSC), for example the calorimeter sold under the name MDSC 2920 by the company TA Instruments.

The measuring protocol is as follows:

A sample of 5 mg of wax placed in a crucible is subjected to a first temperature rise ranging from −20° C. to 100° C., at a heating rate of 10° C./minute, it is then cooled from 100° C. to −20° C. at a cooling rate of 10° C./minute and is finally subjected to a second temperature increase ranging from −20° C. to 100° C. at a heating rate of 5° C./minute. During the second temperature increase, the variation of the difference in power absorbed by the empty crucible and by the crucible containing the sample of wax is measured as a function of the temperature. The melting point of the compound is the temperature value corresponding to the top of the peak of the curve representing the variation in the difference in absorbed power as a function of the temperature.

The waxes that may be used in the compositions according to the invention are chosen from waxes that are solid at room temperature, of animal, plant, mineral or synthetic origin, and mixtures thereof.

The waxes that may be used in the compositions according to the invention generally have a hardness ranging from 0.01 MPa to 15 MPa, especially greater than 0.05 MPa and in particular greater than 0.1 MPa.

The hardness is determined by measuring the compression force, measured at 20° C. using the texturometer sold under the name TA-XT2 by the company Rheo, equipped with a stainless-steel cylindrical spindle 2 mm in diameter, travelling at a measuring speed of 0.1 mm/second, and penetrating the wax to a penetration depth of 0.3 mm.

The measuring protocol is as follows:

The wax is melted at a temperature equal to the melting point of the wax +10° C. The molten wax is poured into a container 25 mm in diameter and 20 mm deep. The wax is recrystallized at room temperature (25° C.) for 24 hours such that the surface of the wax is flat and smooth, and the wax is then stored for at least 1 hour at 20° C. before measuring the hardness or the tack.

The texturometer spindle is displaced at a speed of 0.1 mm/s, then penetrates the wax to a penetration depth of 0.3 mm. When the spindle has penetrated the wax to a depth of 0.3 mm, the spindle is held still for 1 second (corresponding to the relaxation time) and is then withdrawn at a speed of 0.5 mm/s.

The hardness value is the maximum compression force measured divided by the area of the texturometer cylinder in contact with the wax.

As illustrations of waxes that are suitable for the invention, mention may be made especially of hydrocarbon-based waxes, for instance beeswax, lanolin wax and Chinese insect waxes; rice bran wax, carnauba wax, candelilla wax, ouricury wax, alfalfa wax, berry wax, shellac wax, Japan wax and sumach wax; montan wax, microcrystalline waxes, paraffins and ozokerite; polyethylene waxes, the waxes obtained by Fischer-Tropsch synthesis and waxy copolymers, and also esters thereof, waxes obtained by catalytic hydrogenation of animal or plant oils containing linear or branched C₈-C₃₂ fatty chains, for instance hydrogenated jojoba oil, hydrogenated sunflower oil, hydrogenated castor oil, hydrogenated coconut oil, hydrogenated lanolin oil and bis(1,1,1-trimethylolpropane) tetrastearate sold under the name Hest 2T-4S® by the company Heterene.

Mention may also be made of silicone waxes and fluoro waxes.

The waxes obtained by hydrogenation of castor oil esterified with cetyl alcohol, sold under the names Phytowax ricin 16L64® and 22L73® by the company Sophim, may also be used. Such waxes are described in patent application FR-A-2 792 190.

It is also possible to use a “tacky” wax, i.e. a wax with a tack of greater than or equal to 0.7 N.s and a hardness of less than or equal to 3.5 MPa.

Using a tacky wax may especially make it possible to obtain a cosmetic composition that applies easily to the eyelashes, attaches well to the eyelashes and leads to the formation of a smooth, uniform and thickening makeup result.

The tacky wax used may especially have a tack ranging from 0.7 N.s to 30 N.s, in particular greater than or equal to 1 N.s, especially ranging from 1 N.s to 20 N.s, in particular greater than or equal to 2N.s, especially ranging from 2 N.s to 10 N.s and in particular ranging from 2 N.s to 5 N.s.

The tack of the wax is determined by measuring the change in force (compression force or stretching force) as a function of time, at 20° C., using the texturometer sold under the name TA-TX21® by the company Rheo, equipped with a conical acrylic polymer spindle forming an angle of 45°.

The measuring protocol is as follows

The wax is melted at a temperature equal to the melting point of the wax +10° C. The molten wax is poured into a container 25 mm in diameter and 20 mm deep. The wax is recrystallized at room temperature (25° C.) for 24 hours such that the surface of the wax is flat and smooth, and the wax is then stored for at least 1 hour at 20° C. before measuring the tack

The texturometer spindle is displaced at a speed of 0.5 mm/s then penetrates the wax to a penetration depth of 2 mm. When the spindle has penetrated the wax to a depth of 2 mm, the spindle is held still for 1 second (corresponding to the relaxation time) and is then withdrawn at a speed of 0.5 mm/s.

During the relaxation time, the force (compression force) decreases greatly until it becomes zero, and then, during the withdrawal of the spindle, the force (stretching force) becomes negative and then rises again to the value 0. The tack corresponds to the integral of the curve of the force as a function of time for the part of the curve corresponding to negative values of the force (stretching force). The tack value is expressed in N.s.

The tacky wax that may be used generally has a hardness of less than or equal to 3.5 MPa, in particular ranging from 0.01 MPa to 3.5 MPa, especially ranging from 0.05 MPa to 3 MPa or even ranging from 0.1 MPa to 2.5 MPa.

The hardness is measured according to the protocol described previously.

Tacky waxes that may be used include a C₂₀-C₄₀ alkyl(hydroxystearyloxy)stearate (the alkyl group containing from 20 to 40 carbon atoms), alone or as a mixture, in particular a C₂₀-C₄₀ alkyl 12-(12′-hydroxystearyloxy)stearate, of formula (II):

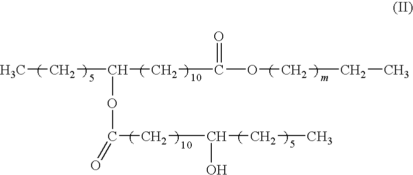

in which m is an integer ranging from 18 to 38, or a mixture of compounds of formula (II).

Such a wax is especially sold under the names Kester Wax K 82 P®, Hydroxypolyester K 82 P® and Kester Wax K 80 P® by the company Koster Keunen.

Waxes provided in the form of small particles having a dimension expressed as the mean “effective” volume diameter D[4.3] of about from 0.5 to 30 micrometres, in particular from 1 to 20 micrometres and more particularly from 5 to 10 micrometres, which are referred to hereinafter as “microwaxes”, may also be used.

The particle sizes may be measured by various techniques; mention may be made in particular of light-scattering techniques (dynamic and static), Coulter counter methods, sedimentation rate measurements (related to the size via Stokes' law) and microscopy. These techniques make it possible to measure a particle diameter and, for some of them, a particle size distribution.

As microwaxes that may be used in the compositions according to the invention, mention may be made of carnauba microwaxes, such as the product sold under the name MicroCare 350® by the company Micro Powders, synthetic microwaxes, such as the product sold under the name MicroEase 114S® by the company Micro Powders, microwaxes consisting of a mixture of carnauba wax and polyethylene wax, such as the products sold under the names Micro Care 300® and 310® by the company Micro Powders, microwaxes consisting of a mixture of carnauba wax and of synthetic wax, such as the product sold under the name Micro Care 325® by the company Micro Powders, polyethylene microwaxes, such as the products sold under the names Micropoly 200®, 220®, 220L® and 250S® by the company Micro Powders, and polytetrafluoroethylene micropowders such as the products sold under the names Microslip 519® and 519 L® by the company Micro Powders.

The wax may represent from 1% to 50% by weight, preferably from 2% to 40% by weight and better still from 5% to 20% by weight relative to the total weight of the first composition.

Pasty Compound

For the purposes of the present invention, the term “pasty” is intended to denote a lipophilic fatty compound that undergoes a reversible solid/liquid change of state and that comprises, at a temperature of 23° C., a liquid fraction and a solid fraction.

A pasty compound is, at a temperature of 23° C., in the form of a liquid fraction and a solid fraction. In other words, the starting melting point of the pasty compound is less than 23° C. The liquid fraction of the pasty compound measured at 23° C. represents from 20% to 97% by weight of the pasty compound. This fraction that is liquid at 23° C. more preferentially represents from 25% to 85% and better still from 30% to 60% by weight of the pasty compound.

The liquid fraction by weight of the pasty compound at 23° C. is equal to the ratio of the heat of fusion consumed at 23° C. to the heat of fusion of the pasty compound.

The heat of fusion consumed at 23° C. is the amount of energy absorbed by the sample to change from the solid state to the state that it has at 23° C., formed from a liquid fraction and a solid fraction.

The heat of fusion of the pasty compound is the heat consumed by the compound to change from the solid state to the liquid state. The pasty compound is said to be in the solid state when all of its mass is in solid form. The pasty compound is said to be in the liquid state when all of its mass is in liquid form.

The heat of fusion of the pasty compound is equal to the area under the curve of the thermogram obtained using a differential scanning calorimeter (DSC), such as the calorimeter sold under the name MDSC 2920 by the company TA Instrument, with a temperature rise of 5 or 10° C. per minute, according to standard ISO 11357-3:1999. The heat of fusion of the pasty compound is the amount of energy required to make the compound change from the solid state to the liquid state. It is expressed in J/g.

The liquid fraction of the pasty compound, measured at 32° C., preferably represents from 40% to 100% by weight of the compound and better still from 50% to 100% by weight of the pasty compound. When the liquid fraction of the pasty compound measured at 32° C. is equal to 100%, the temperature of the end of the melting range of the pasty compound is less than or equal to 32° C.

The liquid fraction of the pasty compound measured at 32° C. is equal to the ratio of the heat of fusion consumed at 32° C. to the heat of fusion of the pasty compound. The heat of fusion consumed at 32° C. is calculated in the same manner as the heat of fusion consumed at 23° C.

The pasty compound preferably has a hardness at 20° C. ranging from 0.001 to 0.5 MPa and preferably from 0.002 to 0.4 MPa.

The hardness is measured according to a method of penetration of a probe into a sample of compound and in particular using a texture analyser (for example the TA-XT2i machine from Rheo) equipped with a stainless-steel cylinder 2 mm in diameter. The hardness measurement is performed at 20° C. at the centre of five samples. The cylinder is introduced into each sample, the penetration depth being 0.3 mm. The hardness value recorded is that of the maximum peak.

The pasty compound may be chosen from synthetic compounds and compounds of plant origin. A pasty compound may be obtained by synthesis from starting materials of plant origin.

The pasty compound is advantageously chosen from:
lanolin and derivatives thereof such as lanolin alcohol, oxyethylenated lanolins, acetylated lanolin, lanolin esters such as isopropyl lanolate, and oxypropylenated lanolins,
polymeric or non-polymeric silicone compounds, for instance polydimethylsiloxanes of high molecular masses, and polydimethylsiloxanes with side chains of the alkyl or alkoxy type containing from 8 to 24 carbon atoms, especially stearyl dimethicones,
polymeric or non-polymeric fluoro compounds,
vinyl polymers, especially:
   olefin homopolymers
   olefin copolymers
   hydrogenated diene homopolymers and copolymers
   linear or branched oligomers, which are homopolymers or copolymers of alkyl(meth)acrylates preferably containing a C₈-C₃₀ alkyl group
   oligomers, which are homopolymers and copolymers of vinyl esters containing C₈-C₃₀ alkyl groups
   oligomers, which are homopolymers and copolymers of vinyl ethers containing C₈-C₃₀ alkyl groups,
liposoluble polyethers resulting from the polyetherification between one or more C₂-C₁₀₀ and preferably C₂-C₅₀ diols,
esters and polyesters,
and mixtures thereof.

The pasty compound may be a polymer and especially a hydrocarbon-based polymer.

A preferred silicone and fluoro pasty compound is polymethyl trifluoropropyl methylalkyl dimethyl-siloxane, manufactured under the name X22-1088 by Shin-Etsu.

When the pasty compound is a silicone and/or fluoro polymer, the composition advantageously comprises a compatibilizer such as short-chain esters, for instance isodecyl neopentanoate.

Among the liposoluble polyethers that that may especially be mentioned are copolymers of ethylene oxide and/or of propylene oxide with C₆-C₃₀ long-chain alkylene oxides. Preferably, the weight ratio of the ethylene oxide and/or of the propylene oxide to the alkylene oxides in the copolymer is from 5:95 to 70:30. In this family, mention will be made especially of block copolymers comprising C₆-C₃₀ alkylene oxide blocks with a molecular weight of from 1000 to 10 000, for example a polyoxyethylene/polydodecylene glycol block copolymer such as the ethers of dodecanediol (22 mol) and of polyethylene glycol (45 oxyethylene or OE units) sold under the brand name Elfacos ST9 by Akzo Nobel.

Among the esters that are especially preferred are:
esters of a glycerol oligomer, especially diglycerol esters, in particular condensates of adipic acid and of glycerol, for which some of the hydroxyl groups of the glycerols have reacted with a mixture of fatty acids such as stearic acid, capric acid, stearic acid and isostearic acid and 12-hydroxystearic acid, for instance those sold under the brand name Softisan 649 by the company Sasol,
phytosterol esters,
pentaerythritol esters,
esters formed from:
   at least one C₁₆₋₄₀ alcohol, at least one of the alcohols being a Guerbet alcohol, and
   a diacid dimer formed from at least one unsaturated C18-40 fatty acid,

for instance the ester of fatty acid dimer of tall oil containing 36 carbon atoms and of a mixture i) of Guerbet alcohols containing 32 carbon atoms and ii) of behenyl alcohol; the ester of linoleic acid dimer and of a mixture of two Guerbet alcohols, 2-tetradecyl-octadecanol (32 carbon atoms) and 2-hexadecyleicosanol (36 carbon atoms),
non-crosslinked polyesters resulting from polycondensation between a linear or branched C₄-C₅₀ dicarboxylic acid or polycarboxylic acid and a C₂-C₅₀ diol or polyol,
polyesters resulting from the esterification, with a polycarboxylic acid, of an aliphatic hydroxy-carboxylic acid ester, for instance Risocast DA-L and Risocast DA-H sold by the Japanese company Kokyu Alcohol Kogyo, which are esters resulting from the esterification reaction of hydrogenated castor oil with dilinoleic acid or isostearic acid,
aliphatic esters of an ester resulting from the esterification of an aliphatic hydroxycarboxylic acid ester with an aliphatic carboxylic acid, for example the product sold under the trade name Salacos HCIS (V)-L sold by the company Nisshin Oil.

A Guerbet alcohol is the reaction product of the Guerbet reaction, which is well known to those skilled in the art. This is a reaction that transforms a primary aliphatic alcohol into its □-alkyl dimer alcohol with loss of one equivalent of water.

The aliphatic carboxylic acids described above generally contain from 4 to 30 and preferably from 8 to 30 carbon atoms. They are preferably chosen from hexanoic acid, heptanoic acid, octanoic acid, 2-ethylhexanoic acid, nonanoic acid, decanoic acid, undecanoic acid, dodecanoic acid, tridecanoic acid, tetradecanoic acid, pentadecanoic acid, hexadecanoic acid, hexyldecanoic acid, heptadecanoic acid, octadecanoic acid, isostearic acid, nonadecanoic acid, eicosanoic acid, isoarachidic acid, octyldodecanoic acid, heneicosanoic acid and docosanoic acid, and mixtures thereof.

The aliphatic carboxylic acids are preferably branched.

The aliphatic hydroxycarboxylic acid esters are advantageously derived from a hydroxylated aliphatic carboxylic acid containing from 2 to 40 carbon atoms, preferably from 10 to 34 carbon atoms and better still from 12 to 28 carbon atoms, and from 1 to 20 hydroxyl groups, preferably from 1 to 10 hydroxyl groups and better still from 1 to 6 hydroxyl groups. The aliphatic hydroxycarboxylic acid esters are especially chosen from:

a) partial or total esters of saturated linear monohydroxylated aliphatic monocarboxylic acids;

b) partial or total esters of unsaturated mono-hydroxylated aliphatic monocarboxylic acids;

c) partial or total esters of saturated mono-hydroxylated aliphatic polycarboxylic acids;

d) partial or total esters of saturated poly-hydroxylated aliphatic polycarboxylic acids;

e) partial or total esters of C₂ to C₁₆ aliphatic polyols that have reacted with a monohydroxylated or polyhydroxylated aliphatic monocarboxylic or poly-carboxylic acid,

f) and mixtures thereof.

The aliphatic esters of an ester are advantageously chosen from:
the ester resulting from the esterification reaction of hydrogenated castor oil with isostearic acid in proportions of 1 to 1 (1/1) or hydrogenated castor oil monoisostearate,

the ester resulting from the esterification reaction of hydrogenated castor oil with isostearic acid in proportions of 1 to 2 (1/2) or hydrogenated castor oil diisostearate,

the ester resulting from the esterification reaction of hydrogenated castor oil with isostearic acid in proportions of 1 to 3 (1/3) or hydrogenated castor oil triisostearate,

and mixtures thereof.

Preferably, the pasty compound is chosen from compounds of plant origin.

Among these compounds, mention may be made especially of isomerized jojoba oil such as the trans-isomerized partially hydrogenated jojoba oil manufactured or sold by the company Desert Whale under the commercial reference Iso-Jojoba-50°, orange wax, for instance the product sold under the reference Orange Peel Wax by the company Koster Keunen, shea butter, for instance the product sold under the reference Beurrolive by the company Soliance, cocoa butter, and mango oil, for instance Lipex 302 from the company Aarhuskarlshamn.

The pasty compound(s) is (are) preferably in an amount of greater than or equal to 1% by weight relative to the total weight of the composition, for example from 1% to 15% by weight, better still in an amount of greater than or equal to 2% by weight, ranging, for example, from 2% to 10% by weight and even more preferentially from 3% to 8% by weight relative to the total weight of the composition.

Non-Silicone Surfactants

The first composition according to the invention may also comprise, besides the silicone surfactant(s), non-silicone emulsifying surfactants present especially in a proportion ranging from 0.1% to 20% and better still from 0.3% to 15% by weight relative to the total weight of the composition.

These surfactants may be chosen from nonionic, anionic, cationic and amphoteric non-silicone surfactants and emulsifying surfactants.

The non-silicone surfactants preferentially used in the composition according to the invention are chosen from:

a) nonionic non-silicone surfactants with an HLB of greater than or equal to 8 at 25° C., used alone or as a mixture; mention may be made especially of:
saccharide esters and ethers such as the mixture of cetylstearyl glucoside and of cetyl and stearyl alcohols, for instance Montanov 68 from SEPPIC;
oxyethylenated and/or oxypropylenated ethers (which may comprise from 1 to 150 oxyethylene and/or oxypropylene groups) of glycerol;
oxyethylenated and/or oxypropylenated ethers (which may comprise from 1 to 150 oxyethylene and/or oxypropylene groups) of fatty alcohols (especially of C8-C24 and preferably C12-C18 alcohol), such as oxyethylenated cetearyl alcohol ether containing 30 oxyethylene groups (CTFA name Ceteareth-30), oxyethylenated stearyl alcohol ether containing 20 oxyethylene groups (CTFA name Steareth-20) and the oxyethylenated ether of the mixture of C12-C15 fatty alcohols comprising 7 oxyethylene groups (CTFA name C12-15 Pareth-7) sold under the name Neodol 25-7® by Shell Chemicals;
fatty acid esters (especially of a C8-C24 and preferably C16-C22 acid) of polyethylene glycol (which may comprise from 1 to 150 ethylene glycol units), such as PEG-50 stearate and PEG-40 monostearate sold under the name Myrj 52P® by the company ICI Uniqema;
fatty acid esters (especially of a C8-C24 and preferably C16-C22 acid) of oxyethylenated and/or oxypropylenated glyceryl ethers (which may comprise from 1 to 250 oxyethylene and/or oxypropylene groups), for instance PEG-200 glyceryl monostearate sold under the name Simulsol 220 TM® by the company SEPPIC; glyceryl stearate polyethoxylated with 30 ethylene oxide groups, for instance the product Tagat S® sold by the company Goldschmidt, glyceryl oleate polyethoxylated with 30 ethylene oxide groups, for instance the product Tagat O® sold by the company Goldschmidt, glyceryl cocoate polyethoxylated with 30 ethylene oxide groups, for instance the product Varionic LI 13° sold by the company Sherex, glyceryl isostearate polyethoxylated with 30 ethylene oxide groups, for instance the product Tagat L® sold by the company Goldschmidt, and glyceryl laurate polyethoxylated with 30 ethylene oxide groups, for instance the product Tagat I® from the company Goldschmidt;
fatty acid esters (especially of a C8-C24 and preferably C16-C22 acid) of oxyethylenated and/or oxypropylenated sorbitol ethers (which may comprise from 1 to 150 oxyethylene and/or oxypropylene groups), for instance polysorbate 20 sold under the name Tween 20® by the company Croda, and polysorbate 60 sold under the name Tween 60® by the company Croda;
copolymers of propylene oxide and of ethylene oxide, also known as EO/PO polycondensates;
and mixtures thereof.

The EO/PO polycondensates are more particularly copolymers consisting of polyethylene glycol and polypropylene glycol blocks, for instance polyethylene glycol/polypropylene glycol/polyethylene glycol triblock polycondensates. These triblock polycondensates have, for example, the following chemical structure:

H—(O—CH₂—CH₂)ₐ—(O—CH(CH₃)—CH₂)_{b}—(O—CH₂—CH₂)ₐ—OH,

in which formula a ranges from 2 to 120 and b ranges from 1 to 100.

The EO/PO polycondensate preferably has a weight-average molecular weight ranging from 1000 to 15 000 and better still ranging from 2000 to 13 000. Advantageously, the EO/PO polycondensate has a cloud point, at 10 g/l in distilled water, of greater than or equal to 20° C. and preferably greater than or equal to 60° C. The cloud point is measured according to ISO standard 1065. As EO/PO polycondensates that may be used according to the invention, mention may be made of the polyethylene glycol/polypropylene glycol/poly-ethylene glycol triblock polycondensates sold under the name Synperonic®, for instance Synperonic PE/L44® and Synperonic PE/F127®, by the company ICI.

b) nonionic non-silicone surfactants with an HLB of less than 8 at 25° C., optionally combined with one or more nonionic surfactants with an HLB of greater than 8 at 25° C., such as those mentioned above, such as:
saccharide esters and ethers, such as sucrose stearate, sucrose cocoate and sorbitan stearate, and mixtures thereof, for instance Arlatone 2121® sold by the company ICI;
oxyethylenated and/or oxypropylenated ethers (which may comprise from 1 to 150 oxyethylene and/or oxypropylene groups) of fatty alcohols (especially of a C8-C24 and preferably C12-C18 alcohol) such as the oxyethylenated ether of stearyl alcohol containing two oxyethylene groups (CTFA name: Steareth-2);
fatty acid esters (especially of a C8-C24 and preferably C16-C22 acid) of polyols, especially of glycerol or of sorbitol, such as glyceryl stearate, glyceryl stearate such as the product sold under the name Tegin M® by the company Goldschmidt, glyceryl laurate such as the product sold under the name Imwitor 312® by the company Hüls, polyglyceryl-2 stearate, sorbitan tristearate or glyceryl ricinoleate;
lecithins, such as soybean lecithins (for instance Emulmetik 100 J from Cargill, or Biophilic H from Lucas Meyer);

c) anionic non-silicone surfactants such as:
C16-C30 fatty acid salts, especially those derived from amines, for instance triethanolamine stearate and/or 2-amino-2-methyl-1,3-propanediol stearate;
polyoxyethylenated fatty acid salts, especially those derived from alkali metal salts, and mixtures thereof;
phosphoric esters and salts thereof, such as DEA oleth-10 phosphate (Crodafos N 10N from the company Croda) or monocetyl monopotassium phosphate (Amphisol K from Givaudan);
sulfosuccinates such as Disodium PEG-5 citrate lauryl sulfosuccinate and Disodium ricinoleamido MEA sulfosuccinate;
alkyl ether sulfates, such as sodium lauryl ether sulfate;
isethionates;
acylglutamates such as Disodium hydrogenated tallow glutamate (Amisoft HS-21 R® sold by the company Ajinomoto) and sodium stearoyl glutamate (Amisoft HS-11 PF® sold by the company Ajinomoto), and mixtures thereof;
soybean derivatives, for instance potassium soyate;
citrates, for instance glyceryl stearate citrate (Axol C 62 Pellets from Degussa);
proline derivatives, for instance sodium palmitoyl proline (Sepicalm VG from SEPPIC) or the mixture of sodium palmitoyl sarcosinate, magnesium palmitoyl glutamate, palmitic acid and palmitoyl proline (Sepifeel One from SEPPIC);
lactylates, for instance sodium stearoyl lactylate (Akoline SL from Karlshamns AB);
sarcosinates, for instance sodium palmitoyl sarcosinate (Nikkol sarcosinate PN) or the 75/25 mixture of stearoyl sarcosine and myristoyl sarcosine (Crodasin SM from Croda);
sulfonates, for instance sodium C14-C17 alkyl sec sulfonate (Hostapur SAS 60 from Clariant);
glycinates, for instance sodium cocoyl glycinate (Amilite GCS-12 from Ajinomoto).

The compositions in accordance with the invention may also contain one or more amphoteric non-silicone surfactants, for instance N-acylamino acids such as N-alkylamino acetates and disodium cocoamphodiacetate and amine oxides such as stearamine oxide.

The surfactant that may be used may also be a polymeric surfactant, especially a heat-gelling polymer.

According to one advantageous embodiment, the first composition according to the invention comprises as additional non-silicone surfactant the following combination:
at least one ether of a C₈-C₂₄ fatty alcohol and of polyethylene glycol, the ether comprising from 1 to 19 oxyethylene units and having an HLB<8 at 25° C.,
at least one ether of a C₈-C₂₄ fatty alcohol and of polyethylene glycol, the ether comprising from 20 to 1000 oxyethylene units and having an HLB>8 at 25° C., and
at least one ester of C₈-C₂₄ fatty acid and of oxyethylenated and/or oxypropylenated glycerol ethers that may comprise from 1 to 250 oxyethylene and/or oxypropylene groups.

Dyestuff

The first composition according to the invention may also comprise at least one dyestuff, for instance pulverulent dyes, liposoluble dyes and water-soluble dyes.

The pulverulent dyestuffs may be chosen from pigments and nacres.

The pigments may be white or coloured, mineral and/or organic, and coated or uncoated. Among the mineral pigments that may be mentioned are titanium dioxide, optionally surface-treated, zirconium oxide, zinc oxide or cerium oxide, and also iron oxide or chromium oxide, manganese violet, ultramarine blue, chromium hydrate and ferric blue. Among the organic pigments that may be mentioned are carbon black, pigments of D&C type, and lakes based on cochineal carmine or on barium, strontium, calcium or aluminium.

The nacres may be chosen from white nacreous pigments such as mica coated with titanium or with bismuth oxychloride, coloured nacreous pigments such as titanium mica with iron oxides, titanium mica with, especially, ferric blue or chromium oxide, titanium mica with an organic pigment of the above-mentioned type, and also nacreous pigments based on bismuth oxychloride.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow and annatto.

These dyestuffs may be present in a content ranging from 0.01% to 30% by weight relative to the total weight of each composition comprising them.

Preferably, the first composition does not comprise any “compound capable of giving the second composition a spinnability dmax of greater than or equal to 5 mm” as defined hereinabove, or comprises such a compound in a content of less than or equal to 1% by weight, preferably less than or equal to 0.5% by weight and better still less than or equal to 0.2% by weight relative to the weight of the first composition.

II) Second Composition

The second composition according to the invention is a composition which, by virtue of its chemical formulation, is difficult to remove.

The term “composition that is difficult to remove” means a composition which, when applied alone to the eyelashes, is not removed using standard makeup removers comprising an aqueous phase, since it may especially comprise film-forming polymers that are insoluble in aqueous solution.

According to a first advantageous embodiment, the second composition comprises an aqueous medium, constituting an aqueous phase, and at least one film-forming polymer dispersed in the aqueous phase in the form of particles as described above for the first composition, the polymer being present in a content at least equal to 5% by weight of solids.

Preferably, when the second composition comprises such a polymer dispersion, it has a continuous aqueous phase.

Preferably, when the second composition comprises such a polymer dispersion, it does not comprise any “compound capable of giving the second composition a spinnability dmax of greater than or equal to 5 mm” as defined hereinabove, or comprises such a compound in a content of less than or equal to 1% by weight, preferably less than or equal to 0.5% by weight and better still less than or equal to 0.2% by weight relative to the weight of the first composition.

Preferably, when the second composition comprises such a polymer dispersion, the first composition, which is applied with the second, does not comprise any dispersion of film-forming polymer in an aqueous phase in the form of particles as defined above, or comprises such a dispersion in a content of less than or equal to 1% by weight of solids, preferably less than or equal to 0.5% by weight of solids and better still less than or equal to 0.2% by weight of solids relative to the weight of the first composition.

According to a second embodiment, the composition comprises at least one liposoluble film-forming polymer. This polymer may be film-forming at room temperature or at a higher temperature not exceeding 100° C.

Examples of liposoluble polymers that may be mentioned are copolymers of vinyl ester (the vinyl group being directly linked to the oxygen atom of the ester group and the vinyl ester containing a saturated, linear or branched hydrocarbon-based radical of 1 to 19 carbon atoms, linked to the carbonyl of the ester group) and of at least one other monomer which may be a vinyl ester (other than the vinyl ester already present), an α-olefin (containing from 8 to 28 carbon atoms), an alkyl vinyl ether (in which the alkyl group comprises from 2 to 18 carbon atoms) or an allylic or methallylic ester (containing a saturated, linear or branched hydrocarbon-based radical of 1 to 19 carbon atoms, linked to the carbonyl of the ester group).

These copolymers may be crosslinked with the aid of crosslinking agents, which may be either of the vinyl type or of the allylic or methallylic type, such as tetraallyloxyethane, divinylbenzene, divinyl octanedioate, divinyl dodecanedioate and divinyl octadecanedioate.

Examples of these copolymers that may be mentioned are the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethyl-propionate/vinyl stearate, allyl dimethylpropion-ate/vinyl stearate, vinyl propionate/vinyl stearate, crosslinked with 0.2% divinylbenzene, vinyl dimethyl-propionate/vinyl laurate, crosslinked with 0.2% divinylbenzene, vinyl acetate/octadecyl vinyl ether, crosslinked with 0.2% tetraallyloxyethane, vinyl acetate/allyl stearate, crosslinked with 0.2% divinylbenzene, vinyl acetate/1-octadecene, crosslinked with 0.2% divinylbenzene, and allyl propionate/allyl stearate, crosslinked with 0.2% divinylbenzene.

Liposoluble film-forming polymers that may also be mentioned include liposoluble copolymers, and in particular those resulting from the copolymerization of vinyl esters containing from 9 to 22 carbon atoms or of alkyl acrylates or methacrylates, and alkyl radicals containing from 10 to 20 carbon atoms.

Such liposoluble copolymers may be chosen from polyvinyl stearate, polyvinyl stearate crosslinked with the aid of divinylbenzene, of diallyl ether or of diallyl phthalate copolymers, polystearyl (meth)acrylate, polyvinyl laurate and polylauryl (meth)acrylate copolymers, it being possible for these poly(meth)acrylates to be crosslinked with the aid of ethylene glycol dimethacrylate or tetraethylene glycol dimethacrylate.

The liposoluble copolymers defined above are known and are described in particular in patent application FR-A-2 232 303; they may have a weight-average molecular weight ranging from 2000 to 500 000 and preferably from 4000 to 200 000.

As liposoluble film-forming polymers that may be used in the invention, mention may also be made of polyalkylenes and in particular copolymers of C2-C20 alkenes, such as polybutene, alkylcelluloses with a linear or branched, saturated or unsaturated C1-C8 alkyl radical, for instance ethylcellulose and propylcellulose, copolymers of vinylpyrrolidone (VP) and in particular copolymers of vinylpyrrolidone and of C2 to C40 and better still C3 to C20 alkene. As examples of VP copolymers which may be used in the invention, mention may be made of the copolymers of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate.

Mention may also be made of silicone resins, which are generally soluble or swellable in silicone oils, which are crosslinked polyorganosiloxane polymers. The nomenclature of silicone resins is known under the name “MDTQ”, the resin being described as a function of the various siloxane monomer units it comprises, each of the letters “MDTQ” characterizing a type of unit.

Examples of commercially available polymethyl-silsesquioxane resins that may be mentioned include those sold:

by the company Wacker under the reference Resin MK, such as Belsil PMS MK;

by the company Shin-Etsu under the reference KR-220L.

Siloxysilicate resins that may be mentioned include trimethyl siloxysilicate (TMS) resins such as those sold under the reference SR1000 by the company General Electric or under the reference TMS 803 by the company Wacker. Mention may also be made of the trimethyl siloxysilicate resins sold in a solvent such as cyclomethicone, sold under the name KF-7312J by the company Shin-Etsu, and DC 749 and DC 593 by the company Dow Corning.

Mention may also be made of copolymers of silicone resins such as those mentioned above with polydimethylsiloxanes, for instance the pressure-sensitive adhesive copolymers sold by the company Dow Corning under the reference Bio-PSA and described in document U.S. Pat. No. 5,162,410, or alternatively silicone copolymers derived from the reaction of a silicone resin, such as those described above, and of a diorganosiloxane as described in document WO 2004/073 626.

Preferably, when the second composition comprises a liposoluble film-forming polymer, it has a continuous oily phase.

Preferably, when the second composition comprises such a film-forming polymer, it does not comprise any “compound capable of giving the second composition a spinnability dmax of greater than or equal to 5 mm” as defined hereinabove, or comprises such a compound in a content of less than or equal to 1% by weight, preferably less than or equal to 0.5% by weight and better still less than or equal to 0.2% by weight relative to the weight of the first composition.

The second composition according to the invention may comprise a plasticizer that promotes the formation of a film with the film-forming polymer. Such a plasticizer may be chosen from any compound known to those skilled in the art as being capable of satisfying the desired function.

According to a third advantageous embodiment, the second composition according to the invention comprises at least one compound capable of giving the second composition a spinnability dmax of greater than or equal to 5 mm.

Preferably, in this case, the second composition according to the invention is anhydrous. The term “anhydrous” means a composition comprising less than 10% by weight and preferably less than 5% by weight of water relative to the total weight of the composition. Preferably, the second composition is free of water.

1) Measurement of the Spinnability

According to a first advantageous embodiment, the second composition according to the invention comprises at least one compound capable of giving the second composition a spinnability dmax of greater than or equal to 5 mm.

The spinnability of the second composition is determined using the texturometer sold under the name TA X-T2i by the company Rheo, equipped with a temperature-regulated spindle, this spindle being a stainless-steel heating cartridge of reference Firerod DIV-STL (from the company Watlow, France), with a diameter of 3.17 mm and a length of 60 mm, a maximum power of 40 W at a voltage of 24V, with a KlocC type thermocouple. The heating cartridge is powered by a 5 V/0.5 A source of DC current, LKS 005-5V from Elka Electronique. Its temperature is regulated by means of a PID TC48 controller from Faucigny Instrument (France). An attachment appendage was created to attach the temperature-regulated spindle to the measuring arm of the texturometer.

The measurement is taken on threads of composition obtained by imposing a vertical displacement of the spindle until it comes into contact with a sample of the composition, and then, after leaving in contact for a moment, imposing an upward vertical displacement of the spindle. Since the composition has spinnability when hot, a thread forms between the spindle during the withdrawal phase and the sample of the composition, this thread becoming more consistent under the effect of cooling in the ambient air. The dmax measurement consists of a measurement of the length of the threads thus formed after detachment from the surface of the spindle.

The protocol is as follows:

a) a sample of the composition is prepared by filling to the brim a stainless-steel crucible 2 mm thick and 20 mm in diameter, the excess composition being shaved off at the surface,

b) the temperature of the spindle is set at 40° C.,

c) the spindle descends at a speed of 10 mm/s until it comes into contact with the surface of the composition,

d) the spindle is held in place for 10 seconds and is then withdrawn at a speed of 10 mm/s.

During the phase of withdrawal of the spindle, a thread is formed between the composition and the spindle. Gradually as the spindle becomes distanced from the surface of the composition, the thread formed cools and becomes more consistent. At a certain elongation, the thread detaches from the spindle.

The spinnability or dmax (expressed in mm) corresponds to the length of the thread obtained after rupture, measured with a graduated ruler.

The spinnability measurement is repeated three times for the same composition, at different places in the crucible, and an average “spinnability” dmax is calculated for each composition.

Steps b) to d) are repeated for the same composition at a spindle temperature set in step b) of, respectively, 50° C., 60° C., 70° C., 80° C., 90° C., 100° C., 110° C., 120° C., 130° C. and 140° C.

Among the spinnability values that may be obtained at the different temperatures, the highest value is selected as the value of the spinnability dmax.

The second composition used in the process according to the invention has a spinnability dmax of greater than or equal to 5 mm, which may be up to 100 mm, preferably greater than or equal to 7 mm, better still greater than or equal to 10 mm and better still greater than or equal to 15 mm.

Preferably, the second composition is capable of forming a thread such that if, after formation of the thread and measurement of the dmax according to the protocol indicated above, the crucible comprising the composition is placed vertically (the thread is in the horizontal position, i.e. subjected to gravity) for at least 30 seconds, the thread maintains a minimum length of 5 mm (which may be measured manually with a graduated ruler).

The second composition having such a spinnability according to the invention allows the production, during application to keratin fibres, of a thread of composition in the prolongation of the eyelash. This thread keeps its shape, remains rigid and does not shrink, which enables a lengthening effect on the eyelash to be obtained.

The second composition used in the process according to the invention is heated to a temperature of greater than or equal to 40° C., preferably greater than or equal to 45° C., better still greater than or equal to 50° C. and even better still greater than or equal to 60° C.

The temperature may be up to 150° C., preferably up to 120° C., better still up to 100° C. and even better still up to 95° C.

According to one particular mode, the second composition is brought to a temperature ranging up to 150° C., preferably up to 120° C., better still up to 100° C. and even better still up to 95° C.

Preferably, the second composition is brought to the temperature at which it shows the spinnability dmax, measured as indicated previously (i.e. to the temperature at which the spinnability is greatest).

2) Compound Capable of Giving the Second Composition a Spinnability dmax of Greater than or Equal to 5 mm

The composition advantageously comprises at least one compound that gives the composition a spinnability dmax of greater than or equal to 5 mm or a mixture of compounds such that the mixture gives the composition a spinnability dmax of greater than or equal to 5 mm, when it is heated to a temperature of greater than or equal to 40° C.

This compound may be a hydrocarbon or silicone compound and advantageously shows thermoplastic behaviour.

This compound is preferably solid at room temperature. Advantageously, it has a spinnability dmax of greater than or equal to 5 mm when it is brought to a temperature of greater than or equal to 40° C., i.e. it is capable of producing threads as described above at a temperature of greater than or equal to 40° C., for example ranging from 40 to 150° C., preferably greater than or equal to 45° C., for example ranging from 45 to 120° C., better still greater than or equal to 50° C., for example ranging from 50 to 100° C. and better still greater than or equal to 60° C.

This compound is preferably a polymer and may be chosen advantageously from:

A/ polymers and copolymers comprising at least one alkene monomer, in particular ethylene-based copolymers.

Such compounds may be chosen from:
copolymers of an alkene and vinyl acetate, preferably copolymers of ethylene and vinyl acetate.

Copolymers of ethylene and vinyl acetate preferably comprising between 5% and 50% by weight, preferably between 10% and 45% by weight and preferably between 20% and 40% by weight of vinyl acetate relative to the total weight of the polymer are used in particular.

Examples of ethylene/vinyl acetate copolymers that may be mentioned include those sold under the name Elvax by the company Du Pont de Nemours and in particular the compounds Elvax 40W, Elvax 140 W, Elvax 200 W, Elvax 205W, Elvax 210 W and Elvax 310.

The products sold under the name Evatane by the company Arkema, such as Evatane 28-800, may also be mentioned. Mention may also be made of Melthene-H Grade H-6410M sold by the company Tosoh Polymer;
copolymers of ethylene and of octene, for instance the products sold under the reference “Affinity” by the company Dow Plastics, for instance Affinity GA 1900 GA 1950;
and mixtures thereof.

These polymers and copolymers may be used alone or as a mixture with at least one compound chosen from tackifying resins as described in the Handbook of Pressure Sensitive Adhesives, edited by Donatas Satas, 3rd edition, 1989, pp. 609-619, waxes as described later, and combinations thereof. The tackifying resins may be chosen especially from rosin, rosin derivatives and hydrocarbon-based resins, and mixtures thereof. Mention may be made in particular of indene-based hydrocarbon-based resins such as resins derived from the polymerization in major proportion of indene monomer and in minor proportion of monomers chosen from styrene, methylindene and methylstyrene, and mixtures thereof. These resins may optionally be hydrogenated. They may have a molecular weight ranging from 290 to 1150. Examples of indene-based resins that may be mentioned in particular include the indene/methyl-styrene/hydrogenated styrene copolymers sold under the name Regalite by the company Eastman Chemical, in particular Regalite R 1100, Regalite R 1090, Regalite R-7100, Regalite R1010Hydrocarbon Resin and Regalite R1125Hydrocarbon Resin.

As mixtures based on ethylene/vinyl acetate copolymer, examples that may be mentioned include the products sold under the name Coolbind by the company National Starch.

These polymers may be in pure form or may be conveyed in an aqueous phase or an organic solvent phase.

B/ Polyvinyl acetate homopolymers, preferably having a molecular weight of less than 20 000, for instance Raviflex BL1S from the company Vinavil.

C/ Silicone resins

These resins are crosslinked organosiloxane polymers.

The nomenclature of silicone resins is known under the name “MDTQ”, the resin being described as a function of the various siloxane monomer units it comprises, each of the letters “MDTQ” characterizing a type of unit.

The letter M represents the monofunctional unit of formula (CH₃)₃SiO_{1/2}, the silicon atom being linked to only one oxygen atom in the polymer comprising this unit.

The letter D means a difunctional unit (CH₃)₂SiO_{2/2} in which the silicon atom is linked to two oxygen atoms.

The letter T represents a trifunctional unit of formula (CH₃) SiO_{3/2}.

In the units M, D and T defined above, at least one of the methyl groups may be substituted with a group R other than a methyl group, such as a hydrocarbon-based radical (especially alkyl) containing from 2 to 10 carbon atoms or a phenyl group, or alternatively a hydroxyl group.

Finally, the letter Q means a tetrafunctional unit SiO_{4/2} in which the silicon atom is linked to four hydrogen atoms, which are themselves linked to the rest of the polymer.

T resins may be mentioned in particular, especially functionalized silicone T resins such as polyphenylsiloxanes, in particular functionalized with silanol groups (Si—OH), such as the product sold under the reference Dow Corning (R) Z-1806.

D/ film-forming block ethylenic polymers

These polymers preferably comprise at least one first block and at least one second block that have different glass transition temperatures (Tg), the first and second blocks being linked together via an intermediate block comprising at least one constituent monomer of the first block and at least one constituent monomer of the second block.

Advantageously, the first and second blocks of the block polymer are mutually incompatible.

Such polymers are described, for example, in document EP 1 411 069 or WO 04/028 488.

The term “block polymer” means a polymer comprising at least two different blocks, for example at least three different blocks.

The first and second blocks of the polymer differ from each other in their degree of deformability. Thus, the first block may be rigid and the second block may be supple.

The glass transition temperatures of the supple and rigid blocks may be theoretical Tg values determined from the theoretical Tg values of the constituent monomers of each of the blocks, which may be found in a reference manual such as the Polymer Handbook, 3rd Edition, 1989, John Wiley, according to the following relationship, known as Fox's law:

1/Tg=Σᵢ( ω̅ᵢ/Tgᵢ),

ω̅ᵢ being the mass fraction of the monomer i in the block under consideration and Tgᵢ being the glass transition temperature of the homopolymer of the monomer i.

Unless otherwise indicated, the Tg values indicated for the first and second blocks in the present patent application are theoretical Tg values.

The rigid block may have a Tg of greater than 20° C.

The supple block may have a Tg of less than or equal to 20° C.

According to one embodiment, the copolymer comprises a first rigid block and a second supple block.

Preferably, the proportion of the rigid block ranges from 20% to 90%, better still from 30% to 90% and even better still from 50% to 90% by weight of the copolymer.

Preferably, the proportion of the supple block ranges from 5% to 75%, preferably from 10% to 50% and better still from 15% to 45% by weight of the copolymer.

Rigid Block

In the context of the present invention, the rigid block(s) is (are) more particularly formed from the following monomers:
methacrylates of formula CH₂═C(CH₃)—COOR₁
   in which R₁ represents a linear or branched unsubstituted C₁ to C₄ alkyl group, such as a methyl, ethyl, propyl or isobutyl group or R₁ represents a C₄ to C₁₂ cycloalkyl group such as an isobornyl group,
acrylates of formula CH₂═CH—COOR₂
   in which R₂ represents a tert-butyl group or a C₄ to C₁₂ cycloalkyl group such as an isobornyl group,
(meth)acrylamides of formula:

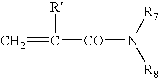

in which R₇ and R₈, which may be identical or different, each represent a hydrogen atom or a linear or branched C₁ to C₁₂ alkyl group such as an n-butyl, t-butyl, isopropyl, isohexyl, isooctyl or isononyl group; or R₇ represents H and R₈ represents a 1,1-dimethyl-3-oxobutyl group,

and R′ denotes H or methyl.

Examples of monomers that may be mentioned include N-butylacrylamide, N-t-butylacrylamide, N-isopropyl-acrylamide, N,N-dimethylacrylamide and N,N-dibutyl-acrylamide,
and mixtures thereof.

Monomers of the rigid block that are particularly preferred are isobornyl methacrylate and isobornyl acrylate, and mixtures thereof.

Supple Block

In the context of the present invention, the supple block(s) is (are) more particularly formed from the following monomers:
the acrylates of formula CH₂═CHCOOR₃, with

R₃ representing a linear or branched C₁ to C₁₂ unsubstituted alkyl group, such as an isobutyl group (with the exception of a tert-butyl group), in which is (are) optionally intercalated one or more heteroatoms chosen from O, N and S,
the methacrylates of formula CH₂═C(CH₃)—COOR₄, with

R₄ representing a linear or branched C₆ to C₁₂ unsubstituted alkyl group, in which is (are) optionally intercalated one or more heteroatoms chosen from O, N and S;
the vinyl esters of formula R₅—CO—O—CH═CH₂ in which R₅ represents a linear or branched C₄ to C₁₂ alkyl group;
C₄ to C₁₂ alkyl vinyl ethers;
and mixtures thereof.

Monomers of the supple block that are particularly preferred are isobutyl acrylate.

Each of the blocks may contain in minor proportion at least one constituent monomer of the other block.

Thus, the first block may contain at least one constituent monomer of the second block, and vice versa.

Each of the first and/or second blocks may comprise, in addition to the monomers indicated above, one or more other monomers known as additional monomers, which are different from the main monomers mentioned previously.

This additional monomer is chosen, for example, from:

a) hydrophilic monomers such as:
ethylenically unsaturated monomers, other than acrylic acid, comprising at least one carboxylic or sulfonic acid function, for instance methacrylic acid, crotonic acid, maleic anhydride, itaconic acid, fumaric acid, maleic acid, acrylamidopropanesulfonic acid, vinylbenzoic acid, vinylphosphoric acid, and salts thereof,
ethylenically unsaturated monomers comprising at least one tertiary amine function, for instance 2-vinylpyridine, 4-vinylpyridine, dimethylaminoethyl methacrylate, diethylaminoethyl methacrylate and dimethylaminopropylmethacrylamide, and salts thereof,
methacrylates of formula CH₂═C(CH₃)—COOR₆

in which R₆ represents a linear or branched alkyl group containing from 1 to 4 carbon atoms, such as a methyl, ethyl, propyl or isobutyl group, the alkyl group being substituted with one or more substituents chosen from hydroxyl groups (for instance 2-hydroxypropyl methacrylate and 2-hydroxyethyl methacrylate) and halogen atoms (Cl, Br, I or F), such as trifluoroethyl methacrylate,
methacrylates of formula CH₂═C(CH₃)—COOR₉,

R₉ representing a linear or branched C₆ to C₁₂ alkyl group in which one or more heteroatoms chosen from O, N and S is (are) optionally intercalated, the alkyl group being substituted with one or more substituents chosen from hydroxyl groups and halogen atoms (Cl, Br, I or F);
acrylates of formula CH₂═CHCOOR₁₀,

R₁₀ representing a linear or branched C₁ to C₁₂ alkyl group substituted with one or more substituents chosen from hydroxyl groups and halogen atoms (Cl, Br, I or F), such as 2-hydroxypropyl acrylate and 2-hydroxyethyl acrylate, or R₁₀ represents a C₁ to C₁₂ alkyl-O—POE (polyoxyethylene) with repetition of the oxyethylene unit 5 to 30 times, for example methoxy-POE, or R₁₀ represents a polyoxyethylenated group comprising from 5 to 30 ethylene oxide units;

b) ethylenically unsaturated monomers comprising one or more silicon atoms, such as methacryloxypropyl-trimethoxysilane and methacryloxypropyltris(trimethyl-siloxy)silane,
and mixtures thereof.

This or these additional monomer(s) generally represent(s) an amount of less than or equal to 30% by weight, for example from 1% to 30% by weight, preferably from 5% to 20% by weight and more preferably from 7% to 15% by weight relative to the total weight of the first and/or second blocks.

According to one embodiment, the copolymer may comprise at least one first block and at least one second block linked together via an intermediate block comprising at least one constituent monomer of the first block and at least one constituent monomer of the second block.

Preferably, the intermediate block is derived essentially from constituent monomers of the first block and of the second block.

Advantageously, the intermediate block comprising at least one constituent monomer of the first block and at least one constituent monomer of the second block of the copolymer is a statistical polymer.

Advantageously, the copolymer is derived essentially from monomers chosen from alkyl methacrylates and alkyl acrylates, and mixtures thereof.

In the text hereinabove and hereinbelow, the term “essentially” means comprising at least 85%, preferably at least 90%, better still at least 95% and even better still 100%. The phrase “consisting essentially of” is not limited by the definition of the term “essentially” herein.

As regards the acrylate and methacrylate esters, they may be derived from the esterification of linear or branched, cyclic or aromatic C₁ to C₁₂ and in particular C₄ to C₁₀ acrylates and methacrylates.

A non-limiting illustration of these alcohols that may especially be mentioned is isoborneol. According to one embodiment, the copolymer comprises at least acrylate and methacrylate monomers derived from the esterification of the same alcohol and in particular of isoborneol.

Preferably, the linear film-forming block polymer comprises at least isobornyl acrylate monomers, at least isobornyl methacrylate monomers and at least isobutyl acrylate monomers.

According to one embodiment variant, the block polymer may comprise at least:
one rigid block, which is an isobornyl methacrylate/isobornyl acrylate copolymer, and
one supple block, which is an isobutyl acrylate copolymer.

More specifically, the copolymer may comprise 50% to 80% by weight of isobornyl methacrylate/acrylate and from 10% to 20% by weight of isobutyl acrylate.

The weight-average mass (Mw) of the copolymer preferably ranges from 80 000 to 300 000 or even from 100 000 to 150 000.

The number-average mass (Mn) of the copolymer preferably ranges from 20 000 to 90 000, for example from 25 000 to 45 000.

E/ Copolymers of diamines and of styrene, especially copolymers of butadiene and/or of styrene.

The styrene/butadiene copolymers sold under the reference Pliolite S5E by the company Eliochem may especially be mentioned.

F/ Polyesters comprising at least one monomer bearing at least one group —SO₃M (M representing a hydrogen atom, an ammonium ion NH₄⁺ or a metal ion), also known as sulfopolyesters.

These polyesters advantageously have a glass transition temperature (Tg) of greater than 38° C.

They may have a weight-average molecular mass advantageously less than 200 000, for example ranging from 10 000 to 50 000.

These polyesters may be obtained, in a known manner, by polycondensation of at least one dicarboxylic acid with at least one polyol, especially diols.

The dicarboxylic acid may be aliphatic, alicyclic or aromatic. Examples of such acids that may be mentioned are: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalene-dicarboxylic acid or 2,6-naphthalenedicarboxylic acid. These dicarboxylic acid monomers may be used alone or as a combination of at least two dicarboxylic acid monomers. Among these monomers, the ones preferentially chosen are phthalic acid, isophthalic acid and terephthalic acid.

The diol may be chosen from aliphatic, alicyclic and aromatic diols. The diol used is preferably chosen from: ethylene glycol, diethylene glycol, triethylene glycol, 1,3-propanediol, cyclohexanedimethanol and 4-butanediol. Other polyols that may be used are glycerol, pentaerythritol, sorbitol and trimethylol-propane.

The polyesteramides may be obtained in a manner analogous to that of the polyesters, by polycondensation of diacids with diamines or amino alcohols.

Diamines that may be used are ethylenediamine, hexamethylenediamine and meta- or para-phenylenediamine. An amino alcohol that may be used is monoethanolamine.

The polyester comprises at least one monomer bearing at least one group —SO₃M, with M representing a hydrogen atom, an ammonium ion NH₄⁺ or a metal ion such as, for example, an Na⁺, Li⁺, K⁺, Mg²⁺, Ca²⁺, Cu²⁺, Fe²⁺ or Fe³⁺ ion. A difunctional aromatic monomer comprising such a group —SO₃M may be used in particular.

The aromatic nucleus of the difunctional aromatic monomer also bearing a group —SO₃M as described above may be chosen, for example, from benzene, naphthalene, anthracene, biphenyl, oxybiphenyl, sulfonylbiphenyl and methylenebiphenyl nuclei. As examples of difunctional aromatic monomers also bearing a group —SO₃M, mention may be made of: sulfoisophthalic acid, sulfotereph-thalic acid, sulfophthalic acid, 4-sulfonaphthalene-2,7-dicarboxylic acid.

The copolymers preferably used are those based on isophthalate/sulfoisophthalate, and more particularly copolymers obtained by condensation of diethylene glycol, cyclohexanedimethanol, isophthalic acid and sulfoisophthalic acid.

Such polymers are sold, for example, under the brand name Eastman AQ® by the company Noveon, for instance Eastman AQ 38S.

G/ Waxes

The waxes may be chosen from the waxes described previously.

Preferably, the wax used in the second composition is a paraffin wax.

H/ Fibres

The term “fibre” should be understood as meaning an object of length L and diameter D such that L is very much greater than D, D being the diameter of the circle in which the cross section of the fibre is inscribed. In particular, the ratio L/D (or shape factor) is chosen in the range from 3.5 to 2500, especially from 5 to 500 and in particular from 5 to 150.

The fibres that may be used in the composition of the invention may be mineral or organic fibres of synthetic or natural origin. They may be short or long, individual or organized, for example braided, and hollow or solid. They may have any shape, and may especially have a circular or polygonal (square, hexagonal or octagonal) cross section, depending on the intended specific application. In particular, their ends are blunt and/or polished to prevent injury.

In particular, the fibres have a length ranging from 1 μm to 10 mm, preferably from 0.1 mm to 5 mm and better still from 0.3 mm to 3.5 mm. Their cross section may be within a circle of diameter ranging from 2 nm to 500 μm, preferably ranging from 100 nm to 100 μm and better still from 1 μm to 50 μm. The weight or yarn count of the fibres is often given in denier or decitex, and represents the weight in grams per 9 km of yarn. In particular, the fibres according to the invention may have a yarn count chosen in the range from 0.15 to 30 denier and better still from 0.18 to 18 denier.

The fibres that may be used in the composition of the invention may be chosen from rigid or non-rigid fibres, and may be of synthetic or natural, mineral or organic origin.

Moreover, the fibres may or may not be surface-treated, may be coated or uncoated, and may be coloured or uncoloured.

As fibres that may be used in the composition according to the invention, mention may be made of non-rigid fibres such as polyamide (Nylon®) fibres or rigid fibres such as polyimideamide fibres, for instance those sold under the names Kermel® and Kermel Tech® by the company Rhodia or poly(p-phenyleneterephthalamide) (or aramid) fibres sold especially under the name Kevlar® by the company DuPont de Nemours.

According to one preferred mode, the second composition comprises at least one compound or mixture of compounds chosen from polymers and copolymers comprising at least one alkene monomer, in particular ethylene-based copolymers.

In particular, the second composition comprises at least one compound or mixture of compounds chosen from copolymers of an alkene and vinyl acetate, in particular copolymers of ethylene and vinyl acetate.

According to another particular mode, the second composition comprises at least one compound or mixture of compounds chosen from copolymers of ethylene and octene.

According to one advantageous embodiment, the second composition comprises at least one ethylene/vinyl acetate copolymer.

According to one advantageous embodiment, the composition comprises, as compounds giving the composition a spinnability dmax of greater than or equal to 5 mm, a mixture of waxes, in particular of paraffin wax and of ethylene/vinyl acetate copolymer.

According to one particular mode, the mixture of wax and of copolymer of ethylene and vinyl acetate comprises from 50% to 65% by weight of ethylene/vinyl acetate copolymer relative to the total weight of the mixture.

According to one particular mode, the mixture of wax and of copolymer of ethylene and vinyl acetate comprises from 35% to 50% by weight of paraffin wax relative to the total weight of the mixture.

The mixture may in particular comprise from 50% to 65% by weight of ethylene/vinyl acetate copolymer relative to the total weight of the mixture and from 35% to 50% by weight of paraffin wax relative to the total weight of the mixture.

Preferably, the ethylene/vinyl acetate copolymer comprises more than 25% by weight, for example about 28% by weight, of vinyl acetate relative to the total weight of the polymer.

Preferably, the ethylene/vinyl acetate copolymer has a weight-average molecular mass (Mw) ranging from 50 000 to 80 000, better still from 60 000 to 70 000 and even better still from 63 000 to 73 000.

The compound or the mixture of compounds giving the composition a spinnability dmax of greater than or equal to 5 mm may be present in the composition in a solids content of at least 5% by weight relative to the total weight of the composition, for example ranging from 5% to 100% by weight, preferably ranging from 10% to 100% by weight and better still from 15% to 100% by weight relative to the total weight of the composition.

3) Other Compounds

The second composition according to the invention may comprise a water-soluble gelling agent.

The water-soluble gelling agents that may be used in the second compositions according to the invention may be chosen from those listed previously for the first composition.

The water-soluble gelling polymer may be present in the second composition comprising it in a solids content ranging from 0.01% to 60% by weight, preferably from 0.5% to 40% by weight and better still from 1% to 30% by weight, or even from 5% to 20% by weight, relative to the total weight of the second composition.

Oils

The first and/or second composition according to the invention may also comprise one or more oils or non-aqueous fatty substances that are liquid at room temperature (25° C.) and atmospheric pressure (760 mmHg).

The oil may be chosen from volatile oils and/or non-volatile oils, and mixtures thereof.

The oil(s) may be present in a content ranging from 0.1% to 95% by weight and preferably from 0.5% to 60% by weight relative to the total weight of the composition.

For the purposes of the invention, the term “volatile oil” means an oil that is capable of evaporating on contact with the skin or the keratin fibre in less than one hour, at room temperature and atmospheric pressure. The volatile organic solvent(s) and volatile oils of the invention are volatile organic solvents and cosmetic oils that are liquid at room temperature, with a non-zero vapour pressure at room temperature and atmospheric pressure, ranging in particular from 0.13 Pa to 40 000 Pa (10⁻³ to 300 mmHg), in particular ranging from 1.3 Pa to 13 000 Pa (0.01 to 100 mmHg), and more particularly ranging from 1.3 Pa to 1300 Pa (0.01 to 10 mmHg).

The term “non-volatile oil” means an oil that remains on the skin or the keratin fibre at room temperature and atmospheric pressure for at least several hours and that especially has a vapour pressure of less than 10⁻³ mmHg (0.13 Pa).

These oils may be hydrocarbon-based oils, silicone oils or fluoro oils, or mixtures thereof.

The term “hydrocarbon-based oil” means an oil mainly containing hydrogen and carbon atoms and optionally oxygen, nitrogen, sulfur or phosphorus atoms. The volatile hydrocarbon-based oils may be chosen from hydrocarbon-based oils containing from 8 to 16 carbon atoms, and especially branched C8-C16 alkanes, for instance C8-C16 isoalkanes of petroleum origin (also known as isoparaffins), for instance isododecane (also known as 2,2,4,4,6-pentamethyl-heptane), isodecane and isohexadecane, for example the oils sold under the trade names Isopar or Permethyl, branched C₈-C₁₆ esters and isohexyl neopentanoate, and mixtures thereof. Other volatile hydrocarbon-based oils, for instance petroleum distillates, especially those sold under the name Shell Solt by the company Shell, may also be used. The volatile solvent is preferably chosen from volatile hydrocarbon-based oils containing from 8 to 16 carbon atoms, and mixtures thereof.

Volatile oils that may also be used include volatile silicones, for instance volatile linear or cyclic silicone oils, especially those with a viscosity ≦8 centistokes (8×10⁻⁶ m²/s) and especially containing from 2 to 7 silicon atoms, these silicones optionally comprising alkyl or alkoxy groups containing from 1 to 10 carbon atoms. As volatile silicone oils that may be used in the invention, mention may be made especially of octamethylcyclotetrasiloxane, decamethyl-cyclopentasiloxane, dodecamethylcyclohexasiloxane, heptamethylhexyltrisiloxane, heptamethyloctyltri-siloxane, hexamethyldisiloxane, octamethyltrisiloxane, decamethyltetrasiloxane and dodecamethylpentasiloxane, and mixtures thereof.

Mention may also be made of linear volatile alkyltrisiloxane oils of general formula (I):

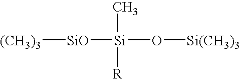

in which R represents an alkyl group containing from 2 to 4 carbon atoms, of which one or more hydrogen atoms may be substituted with a fluorine or chlorine atom.

Among the oils of general formula (I) that may be mentioned are:
3-butyl-1,1,1,3,5,5,5-heptamethyltrisiloxane,
3-propyl-1,1,1,3,5,5,5-heptamethyltrisiloxane, and
3-ethyl-1,1,1,3,5,5,5-heptamethyltrisiloxane,
   corresponding to the oils of formula (I) for which R is, respectively, a butyl group, a propyl group or an ethyl group.

Volatile fluoro solvents such as nonafluoro-methoxybutane or perfluoromethylcyclopentane may also be used.

The first and/or second composition may also comprise at least one non-volatile oil chosen especially from non-volatile hydrocarbon-based oils and/or silicone oils and/or fluoro oils.

Non-volatile hydrocarbon-based oils that may especially be mentioned include:
hydrocarbon-based oils of plant origin, such as triglycerides consisting of fatty acid esters of glycerol, the fatty acids of which may have varied chain lengths from C4 to C24, these chains possibly being linear or branched, and saturated or unsaturated; these oils are especially wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, sesame seed oil, marrow oil, rapeseed oil, blackcurrant oil, evening primrose oil, millet oil, barley oil, quinoa oil, rye oil, safflower oil, candlenut oil, passion flower oil and musk rose oil; or alternatively caprylic/capric acid triglycerides such as those sold by the company Stéarineries Dubois or those sold under the names Miglyol 810, 812 and 818 by the company Dynamit Nobel,
synthetic ethers containing from 10 to 40 carbon atoms;
linear or branched hydrocarbons of mineral or synthetic origin, such as petroleum jelly, polydecenes, hydrogenated polyisobutene such as Parleam, and squalane, and mixtures thereof;
synthetic esters such as oils of formula R1COOR2 in which R1 represents a linear or branched fatty acid residue containing from 1 to 40 carbon atoms and R2 represents an in particular branched hydrocarbon-based chain containing from 1 to 40 carbon atoms, on condition that R1+R2≧10, for instance purcellin oil (cetostearyl octanoate), isopropyl myristate, isopropyl palmitate, C12-C15 alkyl benzoate, hexyl laurate, diisopropyl adipate, isononyl isononanoate, 2-ethylhexyl palmitate, isostearyl isostearate, alkyl or polyalkyl octanoates, decanoates or ricinoleates such as propylene glycol dioctanoate; hydroxylated esters such as isostearyl lactate and diisostearyl malate; and pentaerythritol esters;
fatty alcohols that are liquid at room temperature, containing a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;
higher fatty acids such as oleic acid, linoleic acid or linolenic acid;
carbonates,
acetates,
citrates,

and mixtures thereof.

The non-volatile silicone oils that may be used in the compositions according to the invention may be non-volatile polydimethylsiloxanes (PDMSs), polydimethyl-siloxanes comprising alkyl or alkoxy groups, that are pendent and/or at the end of a silicone chain, the groups each containing from 2 to 24 carbon atoms, phenyl silicones, for instance phenyl trimethicones, phenyl dimethicones, phenyltrimethylsiloxydiphenyl-siloxanes, diphenyl dimethicones, diphenylmethyl-diphenyltrisiloxanes and 2-phenylethyl trimethylsiloxy-silicates.

The fluoro oils that may be used in the invention are, in particular, fluorosilicone oils, fluoro polyethers or fluorosilicones, as described in document EP-A-847 752.

The first and/or second composition may also comprise ingredients commonly used in cosmetics, such as fillers and fibres, and mixtures thereof.

The second composition may also comprise dyestuffs as described above.

Fillers

The first and/or second composition according to the invention may comprise at least one filler.

The fillers may be chosen from those that are well known to those skilled in the art and commonly used in cosmetic compositions. The fillers may be mineral or organic, and lamellar or spherical. Mention may be made of talc, mica, silica, kaolin, polyamide powders, for instance the Nylon® sold under the trade name Orgasol® by the company Atochem, poly-β-alanine powders and polyethylene powders, powders of tetrafluoroethylene polymers, for instance Teflon®, lauroyllysine, starch, boron nitride, expanded polymeric hollow microspheres such as those of polyvinylidene chloride/acrylonitrile, for instance the products sold under the name Expancel® by the company Nobel Industrie, acrylic powders, such as those sold under the name Polytrap® by the company Dow Corning, polymethyl methacrylate particles and silicone resin microbeads (for example Tospearls® from Toshiba), precipitated calcium carbonate, magnesium carbonate and magnesium hydrogen carbonate, hydroxyapatite, hollow silica microspheres (Silica Beads® from Maprecos), glass or ceramic microcapsules, metal soaps derived from organic carboxylic acids containing from 8 to 22 carbon atoms and in particular from 12 to 18 carbon atoms, for example zinc, magnesium or lithium stearate, zinc laurate and magnesium myristate.

It is also possible to use a compound that is capable of swelling on heating, and especially heat-expandable particles such as non-expanded microspheres of copolymer of vinylidene chloride/acrylo-nitrile/methyl methacrylate or of acrylonitrile homopolymer copolymer, for instance those sold, respectively, under the references Expancel® 820 DU 40 and Expancel® 007WU by the company Akzo Nobel.

The fillers may represent from 0.1% to 25% and in particular from 1% to 20% by weight relative to the total weight of the composition.

Fibres

The first and/or second composition according to the invention may comprise, besides the compound or the mixture of compounds that gives the composition a spinnability dmax of greater than or equal to 5 mm, “additional” fibres. These additional fibres alone do not contribute towards the spinnability of the composition.

The additional fibres may be present in the composition according to the invention in a content ranging from 0.01% to 10% by weight, in particular from 0.1% to 5% by weight and more particularly from 0.3% to 3%; by weight relative to the total weight of the composition.

Cosmetic Active Agents

As cosmetic active agents that may be used in the compositions according to the invention, mention may be made especially of antioxidants, preserving agents, fragrances, neutralizers, emollients, moisturizers, vitamins and screening agents, in particular sunscreens.

Needless to say, a person skilled in the art will take care to select the optional additional additives and/or the amount thereof such that the advantageous properties of the compositions according to the invention are not, or are not substantially, adversely affected by the envisaged addition.

The first and/or second compositions may be in solid, semi-solid or liquid form.

The first and/or second compositions may especially be in the form of a suspension, a dispersion, a solution, a gel, an emulsion, especially an oil-in-water (O/W) emulsion, a wax-in-water emulsion or a multiple emulsion (W/O/W or polyol/O/W), in the form of a cream, a paste, a mousse, a dispersion of vesicles, especially of ionic or nonionic lipids, a two-phase or multi-phase lotion, a spray, a powder or a paste, especially a soft paste. Each composition is preferably a leave-in composition.

The second composition may also be in the form of a water-in-oil emulsion (W/O) or a multiple emulsion (O/W/O).

The first and second compositions according to the invention may be manufactured via the known processes generally used in the field of cosmetics.

The first composition may be applied to the eyelashes using a conventional mascara applicator, especially in the form of a brush comprising an arrangement of bristles maintained by a twisted wire. Such a twisted brush is especially described in patent U.S. Pat. No. 4,887,622. It may also be in the form of a comb comprising a plurality of application members, obtained especially by moulding. Such combs are described, for example, in patent FR 2 796 529. The applicator may be solidly attached to the container, as described, for example, in patent FR 2 761 959. Advantageously, the applicator is solidly attached to a stem, which is itself solidly attached to the closing member. In the context of the process according to the invention, the first composition is especially applied to the keratin fibres using a mascara brush.

The first composition is in particular in the form of a wax-in-water emulsion, an aqueous dispersion or an aqueous gel.

In the process of the invention, the second composition, when it comprises at least one compound capable of giving the composition a spinnability dmax of greater than or equal to 5 mm, is generally heated to a temperature of greater than or equal to 40° C., especially greater than or equal to 45° C. and in particular greater than or equal to 50° C.

Obviously, the heating temperature depends in particular on the temperature that the treated support can withstand.

According to one embodiment, the second composition is in solid form.

According to one embodiment, the first composition is in the form of a wax-in-water emulsion, an aqueous dispersion or an aqueous gel, and the second composition is in solid form.

According to a first embodiment of the process according to the invention, the second composition, when it comprises at least one compound capable of giving the composition a spinnability dmax of greater than or equal to 5 mm, is solid and is heated before being applied, the heater used possibly being, or being a part of, the applicator itself. Thus, in the case of a mascara, the second composition may be applied using a heating applicator such as a heating brush.

According to a second embodiment of the process according to the invention, the second composition, when it comprises at least one compound capable of giving the composition a spinnability dmax of greater than or equal to 5 mm, is heated during its application. In such a case, the heater used is generally the applicator itself. Thus, in the case of a mascara, the second composition may be applied using a heating brush.

In a third embodiment, the second composition, when it comprises at least one compound capable of giving the composition a spinnability dmax of greater than or equal to 5 mm, is heated after it has been applied. According to a first variant, the second composition may be heated using something not specifically intended for heating, for instance a body that is occasionally hot. According to a second variant of this embodiment, the composition may be heated using a device specifically dedicated to heating. It may in particular be a device for propelling hot air such as a hairdryer or a drying device, for example as described hereinbelow.

According to one embodiment, the second composition according to the invention, when it comprises at least one compound capable of giving the composition a spinnability dmax of greater than or equal to 5 mm, is in the form of particles, powder or a pulverulent mass. This second composition may be applied to the keratin fibres using an application device comprising a heating support, the composition being contained in an applicator whose shape is adapted to its mounting by slip-fitting onto the heating support, or the composition being contained in a container in which the heating support may be immersed to become charged with composition.

According to one embodiment, the second composition that is in powder form is placed on the heating part of a heating applicator, such as a heating brush or comb, until it softens, and it is then applied to the keratin fibres.

The second composition according to the invention may be conditioned in a packaging and application assembly comprising:

i) a reservoir comprising the composition,

ii) a device for applying the composition; and

iii) heating means or a heater.

According to one embodiment, the heater or heating means are formed by a device that is different from the application device or member, the assembly being configured in the form of a packaging and application device also comprising a container containing a composition in accordance with the invention. Such a device may be conditioned in packaging of the blister pack type. The heater or heating means may be of the type described in patents U.S. Pat. No. 6,009,884 or U.S. Pat. No. 5,853,010. Other devices configured in the form of a heating brush (in the case of the eyelashes) may also be used. Such devices are described especially in patent U.S. Pat. No. 6,220,252.

According to one embodiment, the application device or member comprises a heater or means for heating the second composition; in particular the heater or heating means associated with the application device are arranged so as not to substantially heat at least part of the stem.

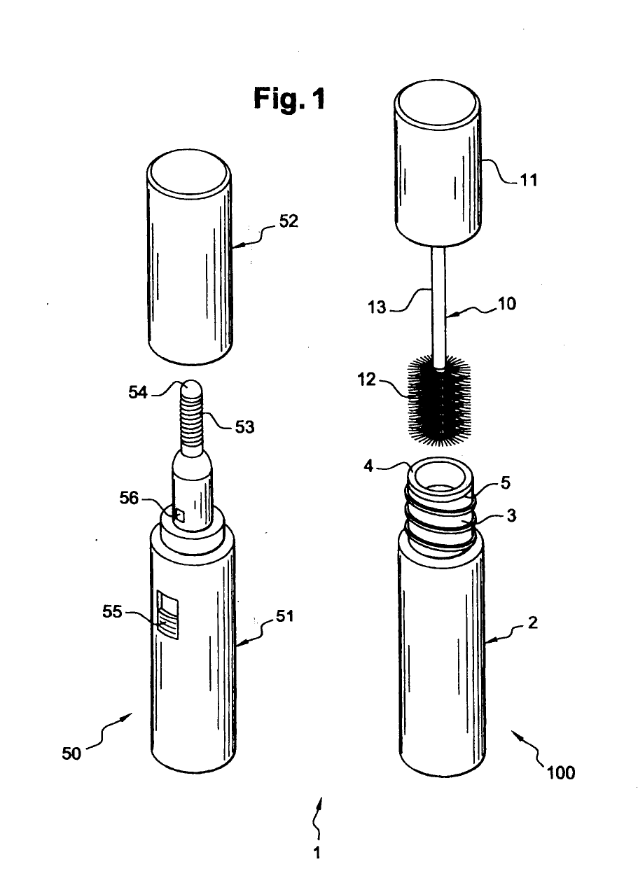

The kit 1 described in FIG. 1 comprises a mascara packaging and application assembly 100 and a heating device 50, separate from the packaging and application assembly.

The two devices 100 and 50 may be sold together in the same packaging, of blister-pack type. The unit 100 containing the product may be sold separately.

The packaging and application assembly 100 comprises a container 2, comprising the composition according to the invention, on which is mounted a threaded collar 3, one free edge of which delimits an opening 4. In the opening 4 is mounted a draining member 5. The assembly 100 also comprises an application device 10 comprising a stopper 11 solidly fastened to a stem 13, one end of which comprises an applicator 12, generally configured in the form of an arrangement of fibres held between the two branches of a twisted iron wire. An inner surface of the stopper 11 is threaded so as to engage with the threading of the neck 3. Thus, when the applicator 12 and the stem 13 are inside the container 2, the threading of the stopper 11 engages with the threading of the neck 3 such that the stopper sealably closes the opening 4 of the container. Such packaging and application assemblies are well known.

The heating device 50 is in accordance with that described in patent U.S. Pat. No. 6,009,884. It mainly comprises a grip portion 51 and a lid 52. A battery is placed inside the grip portion 51 and is connected to a heating wire 53 configured in the form of a coil arranged on a stem 54. A “switch” 55 allows the device to be switched on and off. An LED 56, when it changes colour, indicates that the device is at the required temperature, and is thus ready for use.

The power supply of the heating part via the battery is 12 V. The power dissipated is about 1 watt.

The heating wire 53 may be made of a nickel/chromium alloy.

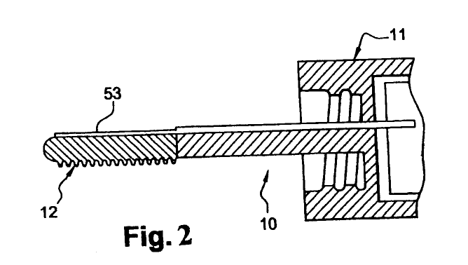

In the embodiment of FIG. 2, the applicator 12 is formed from a metal cylinder, at least part of the periphery of which is striated perpendicular to its longitudinal axis. The striated cylinder is fixed, especially by bonding, to the end of the stem 13. Diametrically opposite, relative to the striated part, is placed a heating resistance 53, which extends over substantially the entire length of the applicator 12. The heating resistance 53 may be placed in a groove made longitudinally in the surface of the cylinder.

Thus, the heating resistance 53 heats the second composition present on the striated cylinder, the striated zone thereof serving for the actual application of the product to the eyelashes, and also for separating them.

According to one embodiment, the second composition is applied cold in a conventional manner to the eyelashes by a brush 12, and is then heated after application: the user engages the heating part 53 of the device 50 onto the eyelashes so as to bring the product deposit to the spinning temperature of the second composition and then drawn out using the heating device formed on the eyelashes so as to create threads in the prolongation of the eyelashes.

On cooling, the threads are set in the prolongation of the eyelashes, allowing a lengthening effect to be obtained.

According to another embodiment, the mascara is in solid form and is used with a heating device 50 alone. It is placed in contact with the heating part 53 of the device 50 and then heated so as to bring the product deposit to the spinning temperature of the second composition. The user then engages the heating part 53 of the device onto the eyelashes and then draws out using the device the deposit formed on the eyelashes so as to create threads in the prolongation of the eyelashes.

The examples given below are presented as non-limiting illustrations of the invention. Unless otherwise mentioned, the amounts indicated are expressed as mass percentages.

### Example 1

### First Compositions

| | Exam- | Example B | | |
|---|---|---|---|---|
| | ple | (compa- | Example C | Example D |
| | A | rative) | (comparative) | (comparative) |
| Acrylamide/ | 5 | 5 | 0 | 0 |
| AMPS | | | | |
| copolymer in | | | | |
| isohexadecane¹ | | | | |
| PEG/ | 5 | 0 | 5 | 0 |
| PPG-17/18 | | | | |
| Dimethicone² | | | | |
| Sodium | 0.75 | 0.75 | 0.75 | 0.75 |
| chloride | | | | |
| Hydroxyethyl- | 2 | 2 | 2 | 2 |
| cellulose | | | | |
| Water | qs | qs 100% | qs 100% | qs 100% |
| | 100% | | | |

¹Simulgel 600 from SEPPIC
²DC Q2-5220 Resin Modifier from Dow Corning

### Second Composition

The second composition is prepared by mixing using an extruder whose cylinders are heated to 100° C.:

| | |
|---|---|
| mixture of paraffin wax and of ethylene/vinyl | 97% |
| acetate copolymer (Coolbind from National Starch) | |
| black iron oxide | 3% |

Each first composition is used for making up three specimens of eyelashes. After drying for 2 minutes, the second composition preheated to 60° C. is applied to the end of the eyelashes, the deposit being drawn out using an applicator to form eyelash extensions.

Each specimen is then pinched in a pad of cotton wool impregnated with Effacil makeup-removing lotion from Lancôme, for 10 seconds, and the cotton wool is then removed.

The percentage of extension of the eyelashes that have been completely freed of makeup is then noted. This operation is performed either 5 minutes after forming the eyelash extensions, or after drying for 24 hours.

| | | Example B | | |
|---|---|---|---|---|
| | Exam- | (compar- | Example C | Example D |
| | ple A | ative) | (comparative) | (comparative) |
| % of eyelashes | 100 | 70 | 70 | 0 |
| freed of | | | | |
| makeup | | | | |
| after 5 minutes | | | | |
| % of eyelashes | 90 | 10 | 50 | 0 |
| freed of | | | | |
| makeup | | | | |
| after 24 hours | | | | |

Application of composition A according to the invention before the second composition allows the eyelashes to be entirely freed of makeup, which is not the case for the Comparative Examples B to D.

### Example 2

### First Compositions

| | Composition E | Composition F | Composition G |
|---|---|---|---|
| Jojoba butter | 6.3 | 6.3 | 6.3 |
| Jojoba oil | 6.3 | 6.3 | 6.3 |
| Carnauba wax | 3.0 | 3.0 | 3.0 |
| Beeswax | 5.5 | 5.5 | 5.5 |
| Steareth-20 | 1.5 | 1.5 | 1.5 |
| Steareth-2 | 1.2 | 1.2 | 1.2 |
| PEG-200 glyceryl | 1.5 | 1.5 | 1.5 |
| stearate | | | |
| Iron oxide | 7.1 | 7.1 | 7.1 |
| Ethanol | 3.0 | 3.0 | 3.0 |
| Propylene glycol | 3.0 | 3.0 | 3.0 |
| Acrylamide/AMPS in | 1.75 | 5 | 5 |
| isohexadecane¹ | | | |
| Sodium chloride | 0 | 0.75 | 0.75 |
| Hydroxyethylcellulose | 0.9 | 0.9 | 0.9 |
| Gum arabic | 3.4 | 3.4 | 3.4 |
| PEG/PPG-17/18 | 5.2 | — | 5.2 |
| Dimethicone² | | | |
| PEG/PPG-16 | — | 1.5 | — |
| dimethicone/ | | | |
| caprylic-capric | | | |
| triglyceride³ | | | |
| Water | qs 100 | qs 100 | qs 100 |

¹Simulgel 600 from SEPPIC
²DC Q2-5220 Resin Modifier from Dow Corning
³Abil Care 85 from Degussa

### Second Composition

The second composition is prepared by mixing using an extruder whose cylinders are heated to 100° C.:

| | |
|---|---|
| mixture of paraffin wax and of ethylene/vinyl | 97% |
| acetate copolymer (Coolbind from National Starch) | |
| black iron oxide | 3% |

Application to the eyelashes of one of the first compositions E to G and then of the second composition makes it possible to obtain an eyelash makeup result that is both charging and very lengthening. The staying power of this makeup is good and its removal is easy using a pad of cotton wool soaked with a makeup-removing lotion such as Effacil from Lancôme.

### Example 3

### Composition Comprising a High Proportion of Film-Forming Agent, and Displaying Easy Removal

| | |
|---|---|
| Jojoba butter | 6.3 |
| Jojoba oil | 6.3 |
| Carnauba wax | 3.0 |
| Beeswax | 5.5 |
| Steareth-20 | 1.5 |
| Steareth-2 | 1.2 |
| PEG-200 glyceryl stearate | 1.5 |
| Iron oxide | 7.1 |
| Ethanol | 3.0 |
| Propylene glycol | 3.0 |
| Acrylamide/AMPS in isohexadecane¹ | 1.75 |
| Sodium chloride | 0 |
| Hydroxyethylcellulose | 0.9 |
| Gum arabic | 3.4 |
| PEG/PPG-16 dimethicone/caprylic-capric | 2 |
| triglyceride³ | |
| Ethyl acrylate/methyl methacrylate copolymer | 20 |
| (80/20) as an aqueous dispersion containing | |
| 50% solids³ | |
| Water | qs 100 |

¹Simulgel 600 from SEPPIC
² Abil Care 85 from Degussa
³Daitosol 5000 AD from Daito

The same composition without acrylamide/AMPS copolymer in isohexadecane and without PEG/PPG-16 dimethicone/caprylic-capric triglyceride is difficult to remove.

### Example 4

### Kit of Composition with a Second Composition Comprising a High Proportion of Film-Forming Agent

| | First | Second |
|---|---|---|
| | composition | composition |
| Acrylamide/AMPS in isohexadecane¹ | 5 | 5 |
| PEG/PPG-17/18 Dimethicone² | 5 | — |
| Sodium chloride | 0.75 | — |
| Hydroxyethylcellulose | 2 | — |
| Styrene-acrylic copolymer as an aqueous | — | 60 |
| emulsion containing 40% solids³ | | |
| Iron oxide | — | 7 |
| Butylene glycol | — | 4 |
| Ethanol | — | 3 |
| Water | qs 100% | qs 100% |

¹Simulgel 600 from SEPPIC
²DC Q2-5220 Resin Modifier from Dow Corning
³Syntran 5760 from Interpolymer

In this example, the second composition applied alone is very difficult to remove at the end of the day, which is not the case for the kit.

### Example 5

### Kit of Composition with a Second Anhydrous Composition Comprising a Liposoluble Film-Forming Polymer

The first composition is identical to that of Example 1 (composition A).

The second composition is as follows:

| | |
|---|---|
| Ethylene/vinyl acetate copolymer (Coolbind from | 2.8 |
| National Starch) | |
| Paraffin wax | 2.2 |
| Synthetic beeswax (Kester Wax K82P from | 26 |
| Koster Keunen; | |
| INCI name: Synthetic beeswax) | |
| vinyl acetate/allyl stearate copolymer (Mexomer PQ | 2.2 |
| from Chimex) | |
| Polyvinyl laurate (Mexomer PP from Chimex) | 0.7 |
| Paraffin wax microbeads (Microease 114S from Micro | 4 |
| Powders) | |
| Propylene carbonate | 0.5 |
| Ethanol | 2.0 |
| Iron oxide | 4.6 |
| Silica | 10 |
| Bentone | 1.5 |
| Isododecane | qs 100 |

This second composition applied alone cannot be removed with a standard aqueous-phase makeup remover such as Effacil from Lancôme.

The combined use of the first and then the second composition allows total makeup removal.

In one embodiment the invention relates to a composition containing a continuous aqueous phase, at least one silicone surfactant and at least one crosslinked polyelectrolyte. Process for making up or caring for keratin fibres, by applying to keratin fibres the noted composition and a second composition different from the first and containing either a continuous aqueous phase and at least one film-forming polymer in the form of particles in dispersion, the polymer being present in an amount at least equal to 5% of solids, or at least one compound or a mixture of compounds which, when the composition is brought to a temperature of greater than or equal to 40° C., gives the composition a spinnability dmax of greater than or equal to 5 mm, the second composition being brought, prior to, simultaneously with or subsequent to its application, to a temperature of greater than or equal to 40° C.

The above written description of the invention provides a manner and process of making and using it such that any person skilled in this art is enabled to make and use the same, this enablement being provided in particular for the subject matter of the appended claims, which make up a part of the original description.

As used herein, the words “a” and “an” and the like carry the meaning of “one or more.”

The phrases “selected from the group consisting of,” “chosen from,” and the like include mixtures of the specified materials. Terms such as “contain(s)” and the like are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A kit comprising:
a first composition comprising a continuous aqueous phase, at least one silicone surfactant and at least one crosslinked polyelectrolyte; and
a second composition, which is different from the first composition, for making up and/or caring for keratin fibres.

2. The kit according to claim 1, wherein the aqueous phase of the first composition represents from 30% to 98% by weight relative to the total weight of the first composition.

3. The kit according to claim 1, wherein the silicone surfactant of the first composition is chosen from nonionic silicone surfactants with an HLB of greater than or equal to 8 at 25° C., amphoteric silicone surfactants, and mixtures thereof.

4. The kit according to claim 1, wherein the silicone surfactant of the first composition is chosen from polydimethylsiloxane containing oxyethylene/oxypropylene end groups as a mixture with caprylic/capric acid triglycerides (INCI name: Bis-PEG/PPG-16/16 PEG/PPG-16/16 dimethicone/caprylic/capric triglyceride), polydimethylsiloxane containing alpha-omega polyether groups (OE/OP:40/60) (INCI name: Bis-PEG/PPG-20/20 dimethicone), oxyethylenated oxypropylenated polydimethylsiloxane (INCI name: PEG/PPG-20/6 dimethicone) and oxyethylenated oxypropylenated polydimethyl/methylsiloxane (INCI name: PEG/PPG-4/12 dimethicone), dimethicone copolyol (INCI name: PEG/PPG-17/18 dimethicone), dimethicone copolyol benzoate, dimethicone copolyol phosphates, and mixtures thereof.

5. The kit according to claim 1, wherein the crosslinked polyelectrolyte of the first composition is chosen from:
partially or totally salified acrylamide/2-methyl-2-[(1-oxo-2-propenyl)amino]-1-propanesulfonic acid (AMPS) copolymer,
crosslinked sodium starch glycolate in powder form,
crosslinked sodium polyacrylates,
polyacrylic acid/alkyl acrylate copolymers,
AMPS,
crosslinked polyoxyethylenated AMPS/alkyl methacrylate copolymers, and
mixtures thereof.

6. The kit according to claim 1, wherein the second composition comprises at least one film-forming polymer in the form of particles dispersed in an aqueous phase, the polymer being present in an amount at least equal to 5% of solids.

7. The kit according to claim 1, wherein the second composition comprises at least one compound or a mixture of compounds, which, when the composition is brought to a temperature of greater than or equal to 40° C., gives the composition a spinnability dmax of greater than or equal to 5 mm,
and wherein the kit further comprises an applicator comprising a heater.

8. The kit according to claim 7, wherein the compound or the mixture of compounds giving the second composition a spinnability dmax of greater than or equal to 5 mm is chosen from:
a) polymers and copolymers comprising at least one alkene monomer,
b) polyvinyl acetate homopolymers,
c) silicone resins,
d) film-forming block ethylenic polymers,
e) copolymers of dienes and of styrene,
f) sulfopolyesters,
g) waxes,
h) fibres,
and mixtures thereof.

9. The kit according to claim 7, wherein the compound or the mixture of compounds giving the second composition a spinnability dmax of greater than or equal to 5 mm is chosen from:
copolymers of an alkene and vinyl acetate,
copolymers of ethylene and octene,
polyvinyl acetate homopolymers,
T silicone resins,
film-forming block ethylenic copolymers comprising one or more polymerized monomers chosen from alkyl methacrylates and alkyl acrylates, and mixtures thereof,
copolymers of butadiene and styrene,
copolymers obtained by condensation of diethylene glycol, cyclohexanedimethanol, isophthalic acid and sulfoisophthalic acid,
and mixtures thereof.

10. The kit according to claim 7, wherein the compound or the mixture of compounds giving the second composition a spinnability dmax of greater than or equal to 5 mm is chosen from mixtures of wax and of a copolymer of ethylene and vinyl acetate.

11. The kit according to claim 7, wherein the compound or the mixture of compounds giving the second composition a spinnability dmax of greater than or equal to 5 mm is present in a solids content ranging from 5% to 100% by weight relative to the total weight of the composition.

12. A process for making up or caring for keratin fibres, comprising the application to the keratin fibres
of a first composition comprising a continuous aqueous phase, at least one silicone surfactant and at least one crosslinked polyelectrolyte;
and then
of a second composition, which is different from the first composition, comprising at least one compound or a mixture of compounds, which, when the second composition is brought to a temperature of greater than or equal to 40° C., gives the second composition a spinnability dmax of greater than or equal to 5 mm,
the second composition being brought, prior to, simultaneously with or subsequent to its application, to a temperature of greater than or equal to 40° C.

13. The process according to claim 12, wherein the keratin fibres are human keratin fibres, and the second composition is applied to the upper end of the keratin fibres, furthest away from the skin, eyelid, or scalp.

14. The process according to claim 12, wherein the second composition is brought, prior to, simultaneously with or subsequent to its application, to a temperature of greater than or equal to 45° C.

15. The process according to claim 12, wherein the keratin fibres are human eyelashes.

16. The process according to claim 13, wherein the keratin fibres are human eyelashes and wherein the second composition is applied to the upper end of the eyelashes, furthest away from the eyelid.

17. A composition comprising a continuous aqueous phase, at least one nonionic silicone surfactant with an HLB of greater than or equal to 8 at 25° C. in a content ranging from 0.2% to 20% by weight relative to the total weight of the composition, and at least one crosslinked polyelectrolyte chosen from acrylamide/2-methyl-2-[(1-oxo-2-propenyl)amino]-1-propanesulfonic acid copolymer, crosslinked sodium starch glycolate in powder form, crosslinked sodium polyacrylates, polyacrylic acid/alkyl acrylate copolymers, crosslinked polyoxyethylenated AMPS/alkyl methacrylate copolymers, and mixtures thereof, and optionally at least 5% by weight of one or more film-forming polymer solids in the form of particles dispersed in an aqueous phase.
